(19) United States
(12) Patent Application Publication
(10) Pub. No.: US 2026/0253741 A1
(43) Pub. Date: Aug. 27, 2026

(54) SYSTEM

(71) Applicant: SoftBank Group Corp., Tokyo (JP)

(72) Inventor: Masayuki ASANO, Tokyo (JP)

(73) Assignee: SoftBank Group Corp., Tokyo (JP)

(21) Appl. No.: 19/542,695

(22) Filed: Feb. 18, 2026

(30) Foreign Application Priority Data
Feb. 21, 2025 (JP) 2025-027001

(51) Int. Cl. G16H 50/30; G06V 10/82; G06V 40/16; G10L 15/16; G10L 25/63; G10L 25/66; G16H 40/67; G16H 50/20; H04W 4/029

(52) U.S. Cl. CPC G16H 50/30; G06V 10/82; G06V 40/174; G10L 15/16; G10L 25/63; G10L 25/66; G16H 40/67; G16H 50/20; H04W 4/029

(57) ABSTRACT

The system according to the embodiment comprises a reception unit, an analysis unit, an evaluation unit, and a connection unit. The reception unit receives images and audio. The analysis unit analyzes the images and audio received by the reception unit. The evaluation unit evaluates a health condition based on a result analyzed by the analysis unit. The connection unit connects to a specific service desk based on a result evaluated by the evaluation unit.

### CROSS-REFERENCE TO RELATED APPLICATION

The present application claims priority to and incorporates by reference the entire contents of Japanese Patent Application No. 2025-027001 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, there has been a problem that it is difficult to quickly and accurately evaluate a child's health condition at home and take appropriate action.

### SUMMARY OF THE INVENTION

The system according to the embodiment comprises a reception unit, an analysis unit, an evaluation unit, and a connection unit. The reception unit receives images and audio. The analysis unit analyzes the images and audio received by the reception unit. The evaluation unit evaluates a health condition based on a result analyzed by the analysis unit. The connection unit connects to a specific service desk based on a result evaluated by the evaluation unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

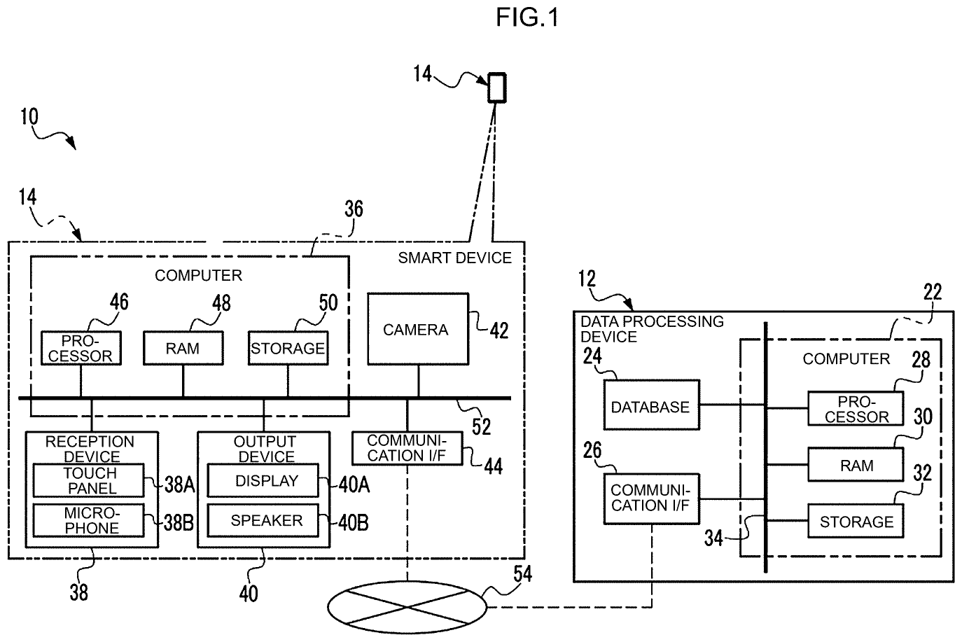

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

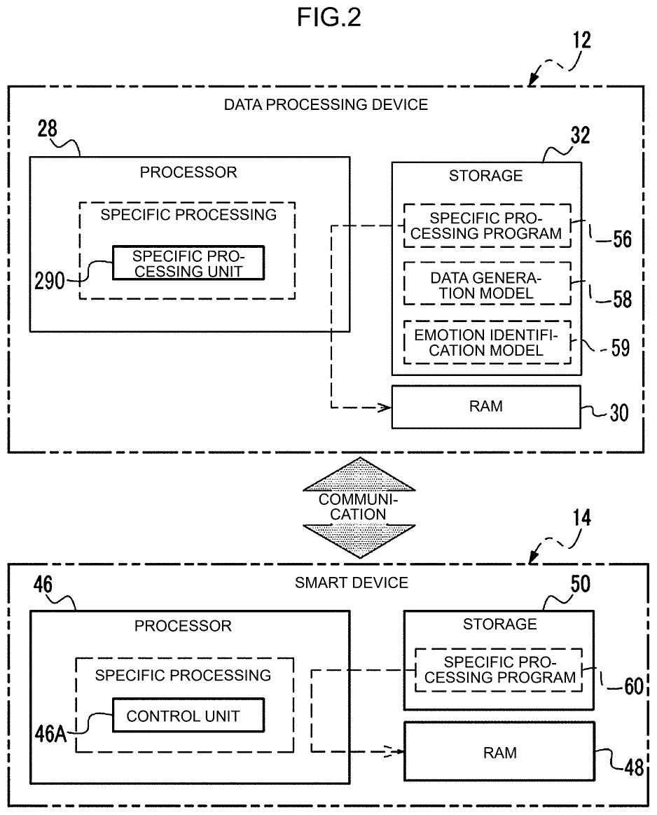

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The health check system according to the embodiment of the present invention is a system that enables health checks for children at home using AI. This health check system allows a parent to send images of the child and audio recordings of the child's breathing to the AI, which then determines the urgency and, if necessary, connects to a dedicated service desk. The AI uses image analysis and speech recognition technology to determine the possibility of illness. Furthermore, by utilizing generative AI, the system can summarize the child's situation and communicate it to a doctor. As the AI learns, it becomes capable of making more advanced judgments, allowing parents to use the system with peace of mind. For example, a parent sends images of the child and audio recordings of the child's breathing to the AI. At this time, the parent can easily capture images and record audio using a smartphone or tablet. For instance, the parent may capture images of the child coughing or looking pale and record the breathing sounds. Next, the AI analyzes the transmitted images and audio. The AI uses image analysis techniques to analyze the child's complexion, facial expressions, and body movements. For example, it can detect cases where the complexion is pale, the facial expression appears distressed, or the body is trembling. Additionally, using speech recognition technology, the AI detects breathing rhythms and abnormal sounds. For example, it can detect cases of labored breathing, severe coughing, or abnormal respiratory sounds. The AI evaluates the health condition based on the analysis results. For example, if the complexion is pale and the breathing is labored, it determines that the situation is urgent. If necessary, the system can connect to a dedicated service desk. For instance, in urgent cases, it connects to an emergency service desk, allowing direct consultation with a doctor. Moreover, the AI uses generative AI to summarize the child's situation and communicate it to the doctor. For example, it summarizes information such as the child's pale complexion and labored breathing and conveys it to the doctor. This enables the doctor to quickly grasp the situation and take appropriate action. As the AI learns, it becomes capable of making more advanced judgments. For example, by learning from past analysis results and medical diagnosis results, the AI improves the accuracy of its judgments. This allows parents to use the system with confidence. As a result, the health check system can quickly and accurately evaluate the child's health condition and take appropriate action as needed. Specifically, the health check system is configured to allow a parent to send image data (e.g., RGB images: resolution 1920×1080 pixels, 8-bit depth) and audio data (e.g., 16 kHz sampling, 16-bit PCM, mono) captured using a smartphone or tablet to the reception unit. The reception unit inputs the image and audio data to respective preprocessing units, performing preprocessing such as noise removal, face region extraction, and color space conversion (e.g., RGB to Lab) for images, and silent segment removal, spectrogram conversion, and extraction of features such as Mel-frequency cepstral coefficients (MFCC) for audio. These preprocessed data are input to neural networks for image analysis (e.g., convolutional neural networks such as ResNet or EfficientNet) and audio analysis (e.g., one-dimensional convolutional networks or LSTM-based time-series models). The image analysis network outputs multiple features such as complexion (e.g., paleness, redness), facial expressions (e.g., distressed, expressionless), and body movements (e.g., trembling, abnormal posture) as multidimensional vectors (e.g., 128-dimensional feature vectors). The audio analysis network detects breathing rhythms (e.g., breathing cycles, inspiration/expiration ratio) and abnormal sounds (e.g., wheezing, coughing, stridor), outputting abnormality scores (e.g., continuous values from 0.0 to 1.0) and labels (e.g., “normal,” “mild abnormality,” “severe abnormality”). These outputs are integrated in the evaluation unit, where health condition is evaluated using methods such as weighted scoring or rule-based threshold judgment (e.g., complexion score <0.3 and breathing abnormality >0.7 for emergency judgment). The evaluation result is output as structured data indicating urgency judgment (e.g., “emergency,” “caution,” “normal”), and the connection unit automatically connects to the appropriate medical service desk (e.g., emergency consultation, general consultation, follow-up observation). Furthermore, the summarization unit inputs the output results from the evaluation unit and features from the analysis unit as prompts to a large language model (e.g., Transformer-based model with billions of parameters) to generate natural language summaries such as “the child's complexion is pale and breathing is labored.” Examples of input to the generative AI include structured text such as “complexion: pale, expression: distressed, breathing sound: labored, cough: present,” and output examples include “This child has a pale complexion, labored breathing, and persistent cough, so emergency response is considered necessary.” These summaries are used for information transmission to medical institutions and feedback to parents. The learning unit of the AI accumulates past image and audio data and medical diagnosis results (e.g., diagnosis labels, treatment progress) as training data, updating weight parameters using error backpropagation based on loss functions (e.g., cross-entropy loss, MSE loss). The training data includes normal and abnormal cases, various ages, and symptom patterns, and data augmentation (e.g., image rotation, brightness changes, audio pitch shift) is also applied. As a result, the AI, unlike conventional human visual inspection, auscultation, and interviews, achieves statistical pattern recognition and multimodal integration in high-dimensional feature space, greatly improving reproducibility, objectivity, and speed of judgment. Technical effects include: (1) convenience for parents to easily check their child's health at home; (2) optimization of medical access through objective and rapid urgency judgment by AI; (3) improved efficiency of information transmission to doctors and diagnostic accuracy; (4) automatic improvement of judgment accuracy through continuous AI learning; (5) reduction of misjudgment risk through integration of diverse data such as images, audio, and text. Specific application fields include home pediatric health management, remote medical support, emergency triage, chronic disease monitoring, health observation in schools and childcare facilities, and early detection support for developmental disorders and respiratory diseases.

The health check system according to the embodiment comprises a reception unit, an analysis unit, an evaluation unit, and a connection unit. The reception unit receives images and audio recorded by a parent. The images and audio recorded by the parent may include, for example, the child's complexion, facial expressions, and breathing sounds, but are not limited to such examples. The reception unit receives images and audio captured using, for example, a smartphone or tablet. Additionally, the reception unit must meet conditions such as image and audio resolution, format, and sound quality. For example, it is desirable for images to be high-resolution and audio to be clear. The analysis unit analyzes the images and audio received by the reception unit. The analysis unit uses image analysis techniques to analyze the child's complexion, facial expressions, and body movements. For example, it can detect cases where the complexion is pale, the facial expression appears distressed, or the body is trembling. The analysis unit also uses speech recognition technology to detect breathing rhythms and abnormal sounds. For example, it can detect cases of labored breathing, severe coughing, or abnormal respiratory sounds. As image analysis techniques, the analysis unit may use face recognition technology or motion analysis technology. As speech recognition technology, it may use speech pattern recognition or abnormal sound detection technology. The evaluation unit evaluates the health condition based on the results analyzed by the analysis unit. The evaluation unit evaluates the child's health condition based on the analysis results. For example, if the complexion is pale and the breathing is labored, it determines that the situation is urgent. The evaluation unit must clarify the evaluation criteria and specific evaluation methods for health condition. For example, it can set evaluation items and scoring methods. The connection unit connects to a dedicated service desk based on the results evaluated by the evaluation unit. The connection unit can connect to a dedicated service desk as needed based on the evaluation results. For example, in urgent cases, it connects to an emergency service desk, allowing direct consultation with a doctor. The connection unit must clarify the specific types and roles of dedicated service desks. For example, it can set medical institutions or consultation desks. As a result, the health check system according to the embodiment can quickly and accurately evaluate the child's health condition and take appropriate action as needed. For example, if a parent feels anxious about the child's health condition, they can quickly send images and audio and receive analysis and evaluation by AI. This allows parents to check their child's health condition with peace of mind. Furthermore, as the AI learns, it becomes capable of making more advanced judgments. For example, by learning from past analysis results and medical diagnosis results, the AI can improve the accuracy of its judgments. As a result, the health check system can always evaluate the child's health condition based on the latest information and take appropriate action. Specifically, the health check system is configured to allow a parent to send image data (e.g., RGB images: resolution 1920×1080 pixels, 8-bit depth) and audio data (e.g., 16 kHz sampling, 16-bit PCM, mono) captured using a smartphone or tablet to the reception unit. The reception unit inputs the image and audio data to respective preprocessing units, performing preprocessing such as noise removal, face region extraction, and color space conversion (e.g., RGB to Lab) for images, and silent segment removal, spectrogram conversion, and extraction of features such as Mel-frequency cepstral coefficients (MFCC) for audio. These preprocessed data are input to neural networks for image analysis (e.g., convolutional neural networks such as ResNet or EfficientNet) and audio analysis (e.g., one-dimensional convolutional networks or LSTM-based time-series models). The image analysis network outputs multiple features such as complexion (e.g., paleness, redness), facial expressions (e.g., distressed, expressionless), and body movements (e.g., trembling, abnormal posture) as multidimensional vectors (e.g., 128-dimensional feature vectors). The audio analysis network detects breathing rhythms (e.g., breathing cycles, inspiration/expiration ratio) and abnormal sounds (e.g., wheezing, coughing, stridor), outputting abnormality scores (e.g., continuous values from 0.0 to 1.0) and labels (e.g., “normal,” “mild abnormality,” “severe abnormality”). These outputs are integrated in the evaluation unit, where health condition is evaluated using methods such as weighted scoring or rule-based threshold judgment (e.g., complexion score <0.3 and breathing abnormality >0.7 for emergency judgment). The evaluation result is output as structured data indicating urgency judgment (e.g., “emergency,” “caution,” “normal”), and the connection unit automatically connects to the appropriate medical service desk (e.g., emergency consultation, general consultation, follow-up observation). Furthermore, the summarization unit inputs the output results from the evaluation unit and features from the analysis unit as prompts to a large language model (e.g., Transformer-based model with billions of parameters) to generate natural language summaries such as “the child's complexion is pale and breathing is labored.” Examples of input to the generative AI include structured text such as “complexion: pale, expression: distressed, breathing sound: labored, cough: present,” and output examples include “This child has a pale complexion, labored breathing, and persistent cough, so emergency response is considered necessary.” These summaries are used for information transmission to medical institutions and feedback to parents. The learning unit of the AI accumulates past image and audio data and medical diagnosis results (e.g., diagnosis labels, treatment progress) as training data, updating weight parameters using error backpropagation based on loss functions (e.g., cross-entropy loss, MSE loss). The training data includes normal and abnormal cases, various ages, and symptom patterns, and data augmentation (e.g., image rotation, brightness changes, audio pitch shift) is also applied. As a result, the AI, unlike conventional human visual inspection, auscultation, and interviews, achieves statistical pattern recognition and multimodal integration in high-dimensional feature space, greatly improving reproducibility, objectivity, and speed of judgment. Technical effects include convenience for parents to easily check their child's health at home, optimization of medical access through objective and rapid urgency judgment by AI, improved efficiency of information transmission to doctors and diagnostic accuracy, automatic improvement of judgment accuracy through continuous AI learning, and reduction of misjudgment risk through integration of diverse data such as images, audio, and text. Specific application fields include home pediatric health management, remote medical support, emergency triage, chronic disease monitoring, health observation in schools and childcare facilities, and early detection support for developmental disorders and respiratory diseases.

The summarization unit can summarize a situation using generative AI. The summarization unit utilizes generative AI to summarize the child's situation and communicate it to a doctor. Generative AI, for example, may use text generation AI (such as LLM) to concisely summarize the child's situation. The summarization unit can also set prompts for summarizing the child's situation using generative AI. For example, a prompt may be set to summarize information such as “the child's complexion is pale and breathing is labored” and input to the generative AI. Based on the prompt, the generative AI can summarize the child's situation and communicate it to the doctor. This enables the summarization unit to quickly and accurately summarize the child's situation and communicate it to the doctor using generative AI. For example, the doctor can quickly grasp the situation and take appropriate action based on the summarized information. Additionally, generative AI can improve the accuracy of summarization through learning. For example, by learning from past summarization results and feedback from doctors, the accuracy of summarization can be improved. As a result, the summarization unit can always summarize the child's situation based on the latest information and communicate it to the doctor. Specifically, the summarization unit is configured to input structured data output from the evaluation unit or analysis unit (e.g., complexion score, breathing abnormality, symptom labels, etc.) as prompts to a large language model (e.g., Transformer-based model with billions of parameters). Examples of input data include JSON format or tagged text such as “complexion: pale, expression: distressed, breathing sound: labored, cough: present.” The generative AI receives these inputs and outputs natural language summaries (e.g., “This child has a pale complexion, labored breathing, and persistent cough, so emergency response is considered necessary.”). The output format can support multiple templates, such as detailed summaries for medical institutions, simplified summaries for parents, and alert messages for emergencies. Furthermore, the summarization unit is equipped with a prompt generation algorithm (e.g., item extraction based on importance scores, template selection by symptom category) that automatically generates optimal prompts according to the content and urgency of the input data. The generative AI is continuously fine-tuned using past summarization results and feedback from doctors (e.g., summary evaluation scores, revision history) as training data. Learning algorithms such as loss functions (e.g., cross-entropy loss, BLEU score optimization) are used to improve summarization accuracy and consistency of expression. As a result, the summarization unit, unlike conventional manual summarization or simple template generation, can automatically generate flexible and highly accurate natural language summaries tailored to diverse symptoms and situations. Technical effects include faster information transmission in medical settings, reduced burden on doctors' decision-making, improved quality of feedback to parents, and continuous improvement of summarization accuracy. Specific application fields include remote medical support, emergency triage, health consultation chatbots, and automatic summarization of medical records.

The learning unit can learn past analysis results or medical diagnosis results. The learning unit learns past analysis results and medical diagnosis results to improve the accuracy of judgment. The learning unit can, for example, collect past analysis results and medical diagnosis results as datasets and use learning algorithms to perform learning. For example, based on past analysis results and medical diagnosis results, the evaluation criteria and methods for health condition can be improved. The learning unit can also refer to past learning data to optimize learning algorithms. For example, it can analyze past learning data and select the optimal learning algorithm. As a result, the learning unit learns past analysis results and medical diagnosis results to improve the accuracy of judgment. For example, by learning past analysis results and medical diagnosis results, the AI becomes capable of making more advanced judgments. This enables the health check system to always evaluate the child's health condition based on the latest information and take appropriate action. Specifically, the learning unit accumulates image data (e.g., RGB images, resolution 1920×1080 pixels), audio data (e.g., 16 kHz sampling, 16-bit PCM), and medical diagnosis labels (e.g., “normal,” “mild abnormality,” “severe abnormality”) and treatment progress information (e.g., number of hospitalization days, prescription information) as training data. Learning algorithms such as convolutional neural networks, recurrent neural networks, and Transformer-based models are used, and weight parameters are updated by error backpropagation based on loss functions (e.g., cross-entropy loss, mean squared error). The training data includes normal and abnormal cases, various ages, and symptom patterns, and data augmentation (e.g., image rotation, brightness changes, audio pitch shift) is also applied. The learning unit also has functions to analyze past learning data, detect overfitting or underfitting of models, and automatically adjust optimal hyperparameters (e.g., learning rate, batch size). Furthermore, the learning unit monitors the output accuracy of the evaluation unit and summarization unit, and realizes continuous model improvement through feedback loops. As a result, the learning unit, unlike conventional human heuristics or simple rule-based processing, achieves statistical pattern recognition and multimodal integrated learning in high-dimensional feature space, greatly improving reproducibility, objectivity, and speed of judgment. Technical effects include automatic improvement of AI judgment accuracy, adaptation to diverse cases, improved quality of diagnostic support in medical settings, and reduction of misjudgment risk. Specific application fields include home health management AI, remote diagnostic support, medical image analysis, audio diagnostic support, and medical data mining.

The reception unit can receive images and audio recorded by a parent under specific conditions. The reception unit receives images and audio recorded by a parent under specific conditions. Specific conditions may include, for example, the shooting environment or recording environment. For example, as a shooting environment, it is desirable to shoot in a bright place or record in a quiet place. As a recording environment, it is desirable to record in a place with little background noise. The reception unit can receive images and audio that meet these conditions. As a result, the reception unit can receive images and audio recorded by a parent under specific conditions. For example, if a parent feels anxious about the child's health condition, they can quickly send images and audio and receive analysis and evaluation by AI. This allows parents to check their child's health condition with peace of mind. Specifically, the reception unit automatically determines conditions such as image data resolution (e.g., 1920×1080 pixels or higher), format (e.g., JPEG, PNG), audio data sampling rate (e.g., 16 kHz or higher), bit depth (e.g., 16-bit), and recording environment noise level (e.g., SNR 30 dB or higher), and provides guidance to the parent to reacquire data if the conditions are not met. The reception unit automatically acquires environmental information at the time of shooting/recording (e.g., illuminance sensor value, microphone input level, estimated background noise) and attaches it as metadata to the image and audio data. Furthermore, the reception unit determines the parent's device model, OS version, camera and microphone performance, and automatically proposes optimal shooting and recording settings. As a result, the reception unit, unlike conventional simple file reception, realizes high-quality data acquisition support functions to maximize AI analysis accuracy. Technical effects include homogenization and improvement of input data quality, stabilization of AI analysis accuracy, reduction of parent operation burden, and reduction of misjudgment risk. Specific application fields include home health management, remote diagnostic support, medical data collection apps, and health observation IoT devices.

The analysis unit can analyze a child's complexion, facial expressions, and body movements using specific image analysis techniques. The analysis unit uses specific image analysis techniques to analyze the child's complexion, facial expressions, and body movements. Specific image analysis techniques may include, for example, face recognition technology and motion analysis technology. For example, face recognition technology can be used to analyze the child's complexion and facial expressions. Motion analysis technology can be used to analyze the child's body movements. The analysis unit can use these technologies to obtain information for evaluating the child's health condition. As a result, the analysis unit can analyze the child's complexion, facial expressions, and body movements using specific image analysis techniques. For example, it can detect cases where the complexion is pale, the facial expression appears distressed, or the body is trembling. This enables the AI to quickly and accurately evaluate the child's health condition. Specifically, the analysis unit receives image data (e.g., RGB images, resolution 1920×1080 pixels, 8-bit depth) captured by a parent using a smartphone or tablet as input. The analysis unit first performs image preprocessing such as noise removal (e.g., median filter), face region extraction (e.g., face detection algorithms such as MTCNN), and color space conversion (e.g., RGB to Lab space). The preprocessed images are input to convolutional neural networks (e.g., ResNet, EfficientNet). The image analysis network extracts multiple features such as complexion (e.g., paleness, redness, jaundice tendency), facial expressions (e.g., distressed, expressionless, smiling), and body movements (e.g., trembling, abnormal posture, limb movements), and outputs them as multidimensional feature vectors of 128 dimensions or more. For example, outputs may include complexion score (0.0-1.0), facial expression label (“distressed,” “normal,” “smiling,” etc.), and body movement abnormality score (0.0-1.0). Examples of output include “complexion score: 0.2, facial expression label: distressed, body movement abnormality: 0.8” or “complexion score: 0.9, facial expression label: normal, body movement abnormality: 0.1.” The analysis unit sends these features to the evaluation unit for use in health condition determination. Face recognition technology may be implemented by combining face detection, landmark extraction, color feature extraction, and facial expression classification (e.g., Softmax classifier). Motion analysis technology may use posture estimation algorithms (e.g., OpenPose) or time-series image feature extraction (e.g., 3D-CNN). As a result, the analysis unit realizes health condition estimation in high-dimensional feature space using statistical and mathematical methods, without relying on human visual inspection or heuristics. Technical effects include: (1) objective and highly reproducible health indicator extraction from image data; (2) reduction of misjudgment risk through simultaneous analysis of multiple features; (3) rapid automatic judgment for faster medical access; (4) improved adaptability to diverse symptom patterns. Specific application fields include home health management, remote diagnostic support, emergency triage, health observation in schools and childcare facilities, and early detection support for developmental and neurological disorders.

The analysis unit can detect breathing rhythms and abnormal sounds using speech recognition technology. The analysis unit uses speech recognition technology to detect breathing rhythms and abnormal sounds. Speech recognition technology may include, for example, speech pattern recognition and abnormal sound detection technology. For example, speech pattern recognition can be used to analyze breathing rhythms. Abnormal sound detection technology can be used to detect abnormal breathing sounds. The analysis unit can use these technologies to obtain information for evaluating the child's health condition. As a result, the analysis unit can detect breathing rhythms and abnormal sounds using speech recognition technology. For example, it can detect cases of labored breathing, severe coughing, or abnormal respiratory sounds. This enables the AI to quickly and accurately evaluate the child's health condition. Specifically, the analysis unit receives audio data (e.g., 16 kHz sampling, 16-bit PCM, mono) recorded by a parent using a smartphone or tablet as input. The analysis unit first performs audio preprocessing such as silent segment removal (e.g., energy threshold method), noise reduction (e.g., spectral subtraction), and volume normalization. The preprocessed audio waveform undergoes feature extraction processing such as spectrogram conversion (e.g., STFT for time-frequency representation) and Mel-frequency cepstral coefficient (MFCC) extraction, and is input to neural networks for audio analysis (e.g., one-dimensional convolutional networks, LSTM-based time-series models, or Transformer-based acoustic models). The audio analysis network extracts multiple features such as breathing rhythms (e.g., breathing cycles, inspiration/expiration ratio, variation in breathing intervals), abnormal sounds (e.g., wheezing, coughing, stridor, apnea segments), and outputs abnormality scores (e.g., continuous values from 0.0 to 1.0), abnormal sound labels (e.g., “normal,” “mild abnormality,” “severe abnormality”), and timestamps of abnormal sound occurrences. Examples of input include “16 kHz, 10-second breathing sound waveform” or “breathing sound with cough,” and output examples include “abnormality score: 0.85, abnormal sound label: severe abnormality, cough occurrence timestamps: 2.3 s, 7.8 s” or “abnormality score: 0.15, abnormal sound label: normal.” The analysis unit sends these outputs to the evaluation unit, where they are integrated with image analysis results for health condition determination. Abnormal sound detection technology may be implemented by combining spectral pattern matching, autoencoder-based anomaly detection, or supervised classification models (e.g., CNN+LSTM hybrid). Furthermore, the analysis unit can perform time-series analysis of audio data and comparative analysis of multiple recordings, enabling detection of sudden symptom deterioration or chronic abnormalities. As a result, the analysis unit realizes health condition estimation in high-dimensional feature space using statistical and mathematical methods, without relying on human auscultation or heuristics. Technical effects include: (1) objective and highly reproducible health indicator extraction from audio data; (2) reduction of misjudgment risk through simultaneous analysis of multiple features; (3) rapid automatic judgment for faster medical access; (4) improved adaptability to diverse symptom patterns; (5) enhanced robustness to variations in audio data quality. Specific application fields include home health management, remote diagnostic support, emergency triage, early detection of respiratory diseases, health observation in schools and childcare facilities, chronic disease monitoring, and detection of vocal signs of developmental and neurological disorders.

The evaluation unit can evaluate a health condition based on the analysis result. The evaluation unit evaluates the child's health condition based on the analysis result. For example, if the complexion is pale and the breathing is labored, it determines that the situation is urgent. The evaluation unit must clarify the evaluation criteria and specific evaluation methods for health condition. For example, it can set evaluation items and scoring methods. The evaluation unit can evaluate the child's health condition based on these criteria. As a result, the evaluation unit can quickly and accurately evaluate the child's health condition based on the analysis result. For example, the AI evaluates the child's health condition based on the analysis result and can take appropriate action as needed. Specifically, the evaluation unit implements an algorithm for quantitatively evaluating health condition by integrating multidimensional feature vectors (e.g., complexion score, facial expression label, body movement abnormality, breathing abnormality score, abnormal sound label, etc.) received from the image analysis unit and audio analysis unit. The evaluation unit first sets weighting parameters for each feature (e.g., complexion 0.4, breathing abnormality 0.4, body movement 0.2) and calculates a comprehensive score (e.g., continuous value from 0.0 to 1.0). The evaluation unit can apply rule-based threshold judgment logic, such as emergency judgment for “complexion score <0.3 and breathing abnormality >0.7,” caution judgment for “complexion score 0.3-0.6 and breathing abnormality 0.4-0.7,” and normal judgment for “complexion score >0.6 and breathing abnormality <0.4.” Examples of input data include “complexion score: 0.2, breathing abnormality: 0.85, facial expression label: distressed, body movement abnormality: 0.8” or “complexion score: 0.9, breathing abnormality: 0.1, facial expression label: normal, body movement abnormality: 0.1.” Examples of output data include health condition labels such as “emergency,” “caution,” “normal,” and urgency scores (e.g., 0.92) as structured data. The evaluation unit sends these outputs to the connection unit and summarization unit for subsequent medical service desk connection and natural language summarization generation. Furthermore, the evaluation unit can receive feedback from past evaluation results and medical diagnosis results and automatically optimize evaluation criteria and weighting parameters (e.g., Bayesian optimization, grid search). As a result, the evaluation unit, unlike conventional subjective evaluation or simple rule-based processing by humans, realizes statistical pattern recognition and multimodal integrated evaluation in high-dimensional feature space, greatly improving reproducibility, objectivity, and speed of judgment. Technical effects include: (1) optimization of medical access through objective and rapid health condition evaluation by AI; (2) continuous improvement of accuracy through automatic optimization of evaluation criteria; (3) reduction of misjudgment risk through integration of diverse data such as images, audio, and text; (4) automation of subsequent processing through structured evaluation results. Specific application fields include home health management AI, remote diagnostic support, emergency triage, chronic disease monitoring, health observation in schools and childcare facilities, and early detection support for developmental and respiratory diseases.

The connection unit can connect to a dedicated service desk based on the evaluation result. The connection unit connects to a dedicated service desk based on the evaluation result. For example, in urgent cases, it connects to an emergency service desk, allowing direct consultation with a doctor. The connection unit must clarify the specific types and roles of dedicated service desks. For example, it can set medical institutions or consultation desks. The connection unit can quickly connect to these service desks. As a result, the connection unit can quickly connect to a dedicated service desk based on the evaluation result. For example, the AI can connect to the appropriate service desk according to the child's health condition based on the evaluation result. This enables parents to quickly consult with a doctor and receive appropriate care. Specifically, the connection unit receives health condition labels (e.g., “emergency,” “caution,” “normal”) and urgency scores (e.g., continuous value from 0.0 to 1.0) from the evaluation unit as input. The connection unit first analyzes the evaluation result and generates a candidate list of connection destinations based on pre-set connection rules (e.g., urgency score >0.8 for emergency consultation desk, 0.5-0.8 for general consultation desk, less than 0.5 for follow-up observation desk). The connection unit obtains attribute information of each service desk (e.g., available hours, specialties, congestion status, communication method) from a database and selects the optimal connection destination. For example, in urgent cases, it prioritizes emergency consultation desks available 24 hours and automatically selects dedicated medical institution lines or video call APIs. The connection unit automatically executes connection procedures to the selected service desk (e.g., API calls, session generation, authentication information assignment, communication channel establishment) and immediately sends connection completion notifications or connection links to the parent's device. Furthermore, the connection unit records connection history and past response results for use in optimizing future connections and strengthening collaboration with medical institutions. The connection unit also has fail-safe functions to automatically reselect alternative service desks and provide retry processing or re-guidance to parents in case of connection failure. Examples of AI evaluation result outputs include labels such as “emergency,” “caution,” “normal,” and urgency scores (e.g., 0.92), which serve as input to the connection unit. Examples of connection unit outputs include specific actions such as “video call connection to emergency consultation desk,” “chat connection to general consultation desk,” and “reminder setting for follow-up observation.” Subsequent processing includes information transfer to the connected medical institution, notification of connection status to the parent, and logging of connection sessions. Technical effects include: (1) faster medical access through automation of health condition evaluation and medical service desk connection by AI; (2) optimal allocation of medical resources through matching of evaluation results and service desk attributes; (3) continuous improvement of connection accuracy through accumulation of connection history; (4) improved connection reliability through fail-safe functions. Specific application fields include home health management AI, remote diagnostic support, emergency triage, chronic disease monitoring, health observation in schools and childcare facilities, early detection support for developmental and respiratory diseases, and automatic patient routing systems for medical institutions.

The reception unit can estimate a parent's emotion and adjust the timing of acquiring images and audio based on the estimated emotion. The reception unit estimates a parent's emotion and adjusts the timing of acquiring images and audio based on the estimated emotion. To estimate a parent's emotion, for example, facial expression recognition or audio analysis technology can be used. For example, if the parent is anxious, the system can prompt immediate acquisition of images and audio. If the parent is relaxed, the system can propose an appropriate timing for acquisition. Furthermore, if the parent is busy, the system can set a reminder for later acquisition. As a result, the reception unit can adjust the timing of acquiring images and audio based on the parent's emotion. For example, if the parent is anxious, images and audio can be acquired quickly and analyzed and evaluated by AI. This allows parents to check their child's health condition with peace of mind. Specifically, the reception unit is configured to simultaneously acquire, in addition to image data (e.g., RGB images, 1920×1080 pixels, 8-bit depth) and audio data (e.g., 16 kHz sampling, 16-bit PCM) obtained from the parent's device, the parent's facial image (e.g., face image from the front camera) and audio input (e.g., speech content, voice tone). The reception unit uses a facial expression recognition module (e.g., convolutional neural network-based facial expression classifier) to estimate emotion labels such as “anxious,” “relaxed,” or “busy” from the parent's facial image. The audio analysis module extracts features such as MFCC, pitch, speech rate, and voice intensity from the parent's speech audio and estimates emotion using recurrent neural networks or Transformer-based models. Examples of input include “parent's facial image (expression: furrowed brow, downturned mouth corners)” and “parent's audio (high pitch, fast speech rate),” and output examples include “emotion label: anxious,” “emotion score: 0.85.” Based on the estimated emotion label and score, the reception unit's image/audio acquisition timing control module automatically determines actions such as immediate acquisition instructions, acquisition timing proposals, or reminder settings. For example, “if emotion score is 0.8 or higher and anxious, prompt immediate acquisition,” “if emotion score is less than 0.3 and relaxed, propose acquisition timing to the parent,” “if emotion score is 0.5 or higher and busy, automatically set a reminder.” These controls are executed as push notifications or guidance displays on the parent's device. Subsequent processing involves sending the acquired image and audio data to the preprocessing and analysis units for AI-based health condition analysis. Technical effects include: (1) improved quality and freshness of input data through optimal acquisition timing control according to the parent's psychological state; (2) reduced burden on parents and increased system usage rate; (3) improved accuracy and reliability of AI analysis; (4) realization of personalized interaction, unlike conventional uniform data acquisition instructions. Specific application fields include home health management AI, remote diagnostic support, child monitoring IoT, mental health care support, and stress detection-based health management in care settings.

The reception unit can analyze a parent's past submission history of images and audio and select an appropriate acquisition method. The reception unit analyzes a parent's past submission history of images and audio and selects an appropriate acquisition method. For example, the system can preferentially propose devices previously used by the parent. It can also propose acquisition timing based on the time periods when the parent previously submitted data. Furthermore, by analyzing the quality of images and audio previously submitted by the parent, the system can propose the optimal acquisition method. As a result, the reception unit can analyze a parent's past submission history of images and audio and select an appropriate acquisition method. For example, by referring to devices and time periods previously used by the parent, the system can acquire images and audio in the optimal way for the parent. This allows parents to efficiently submit images and audio and receive analysis and evaluation by AI. Specifically, the reception unit manages a database of submission history for each parent (e.g., submission date and time, device model used, OS version, image resolution, audio sampling rate, network environment at submission, image/audio quality score, etc.). The reception unit uses a history analysis module (e.g., time-series clustering algorithm, decision tree-based pattern extractor) to automatically extract submission tendencies for each parent (e.g., submissions from a smartphone at night on weekdays, submissions from a tablet during the day on holidays) and past submission quality (e.g., image blur rate, audio noise level). Examples of input include “Parent A: past 10 submissions (device: smartphone 8 times, tablet 2 times; time period: 7 times in the 8 pm hour; average quality score 0.92),” and output examples include “recommended device: smartphone; recommended time period: 8 pm hour; recommended acquisition method: high resolution, noise reduction settings.” Based on these analysis results, the reception unit provides guidance to the parent's device on the optimal acquisition method (e.g., recommended device selection, recommended shooting/recording settings, recommended acquisition timing). Furthermore, if past submission quality is low, the system automatically generates suggestions for points to note during shooting/recording or for reacquisition. Subsequent processing involves sending images and audio data acquired using the optimized method to the preprocessing and analysis units, contributing to improved accuracy of AI-based health condition analysis. Technical effects include: (1) personalized data acquisition support based on each parent's usage tendencies and history; (2) homogenization and improvement of input data quality; (3) stabilization of AI analysis accuracy; (4) reduced burden on parents and increased system usage rate; (5) history-based optimization, unlike conventional uniform acquisition instructions. Specific application fields include home health management AI, remote diagnostic support, medical data collection apps, health observation IoT devices, and history-based optimized health management in care and welfare settings.

The reception unit can perform filtering based on a parent's current living situation and areas of interest when acquiring images and audio. The reception unit performs filtering based on a parent's current living situation and areas of interest when acquiring images and audio. For example, if the parent is at work, the system can propose refraining from acquisition. If the parent is interested in the child's health, the system can propose detailed acquisition methods. Furthermore, if the parent is traveling, the system can propose postponing acquisition. To identify the parent's current living situation and areas of interest, for example, survey results or past behavioral history can be used. As a result, the reception unit can filter image and audio acquisition based on the parent's current living situation and areas of interest. For example, by proposing to refrain from acquisition when the parent is at work, the system can reduce the parent's burden. This allows parents to efficiently submit images and audio and receive analysis and evaluation by AI. Specifically, the reception unit manages a database of the parent's living situation and areas of interest (e.g., occupation, working hours, hobbies, health interest level, travel plans, past behavioral history, survey responses, etc.). The reception unit uses a living situation estimation module (e.g., calendar integration API, location information analysis, device usage monitoring) and an interest estimation module (e.g., past health-related app usage history, survey score analysis) to estimate the parent's current situation and interest level in real time. Examples of input include “Parent B: working hours 9 am-6 pm on weekdays, health interest score 0.95, travel plans: this weekend,” and output examples include “recommended acquisition timing: outside working hours; acquisition method: with detailed guidance; acquisition postponement proposal: during travel period.” Based on these estimation results, the reception unit automatically adjusts the timing and method of image and audio acquisition and provides optimal acquisition guidance or reminders to the parent's device. Furthermore, if the parent's interest level is high, the system also provides detailed acquisition procedures and health management information. Subsequent processing involves sending data acquired at filtered timing and by filtered methods to the preprocessing and analysis units, contributing to improved accuracy of AI analysis and increased parent satisfaction. Technical effects include: (1) flexible data acquisition control according to the parent's living situation and interest level; (2) reduced burden on parents and increased system usage rate; (3) stable acquisition of high-quality, high-interest data for AI analysis; (4) realization of situation-adaptive interaction, unlike conventional uniform acquisition instructions. Specific application fields include home health management AI, remote diagnostic support, health observation IoT devices, work-life balance-conscious health management, and personalized health support services.

The reception unit can estimate a parent's emotion and determine the priority of images and audio to be acquired based on the estimated emotion. The reception unit estimates a parent's emotion and determines the priority of images and audio to be acquired based on the estimated emotion. To estimate a parent's emotion, for example, facial expression recognition or audio analysis technology can be used. For example, if the parent is anxious, images and audio can be acquired immediately. If the parent is relaxed, acquisition can be performed at an appropriate timing. Furthermore, if the parent is busy, the system can set a reminder for later acquisition. As a result, the reception unit can determine the priority of images and audio to be acquired based on the parent's emotion. For example, if the parent is anxious, images and audio can be acquired quickly and analyzed and evaluated by AI. This allows parents to check their child's health condition with peace of mind. Specifically, the reception unit estimates emotion labels and scores (e.g., “anxious” 0.85, “relaxed” 0.2, “busy” 0.7, etc.) from the parent's facial image and audio input and inputs them to the image/audio acquisition priority determination module. The priority determination module automatically adjusts the immediacy and order of image and audio acquisition according to the emotion score. For example, rules or machine learning-based priority determination algorithms are applied, such as “if anxiety score is 0.8 or higher, prioritize immediate acquisition of both images and audio,” “if relaxation score is high, propose acquisition timing to the parent,” “if busy score is high, prioritize reminder setting.” Examples of input include “emotion label: anxious, score: 0.9,” “emotion label: busy, score: 0.7,” and output examples include “image/audio acquisition priority: high,” “reminder setting: enabled.” The reception unit immediately notifies the parent's device of acquisition instructions or guidance according to the priority, and sends the acquired data to the preprocessing and analysis units. Subsequent processing involves rapid evaluation of high-priority data by AI analysis, optimizing emergency response and feedback to parents. Technical effects include: (1) improved emergency response capability through data acquisition priority control according to the parent's psychological state; (2) rapid acquisition of important data for AI analysis; (3) increased parent satisfaction and peace of mind; (4) realization of personalized priority control, unlike conventional uniform acquisition order. Specific application fields include home health management AI, remote diagnostic support, emergency response-type health observation, stress detection-type health management, and priority control-type data acquisition in care settings.

The reception unit can preferentially acquire highly relevant information based on a parent's geographic location when acquiring images and audio. The reception unit preferentially acquires highly relevant information based on a parent's geographic location when acquiring images and audio. For example, if the parent is at home, the system can prioritize acquisition of indoor images and audio. If the parent is outside, the system can consider ambient environmental sounds during acquisition. Furthermore, if the parent is traveling, the system can consider information about the travel destination during acquisition. To obtain the parent's geographic location, for example, GPS data or address information can be used. As a result, the reception unit can preferentially acquire highly relevant information based on the parent's geographic location. For example, by prioritizing acquisition of indoor images and audio when the parent is at home, the system can accurately evaluate the child's health condition. This allows parents to efficiently submit images and audio and receive analysis and evaluation by AI. Specifically, the reception unit automatically acquires geographic location information from the parent's device, such as GPS data (e.g., latitude, longitude, location accuracy), Wi-Fi/Bluetooth beacon information, and address information. The location information analysis module classifies the acquired location information into categories such as “home,” “outside,” or “travel destination,” and determines the most relevant image and audio acquisition method. For example, when “home” is detected, the system prioritizes use of indoor cameras and microphones; when “outside” is detected, it prompts settings for environmental noise reduction and input of surrounding situation descriptions; when “travel destination” is detected, it reflects local environmental information (e.g., temperature, humidity, infectious disease prevalence, etc.) in the acquisition guidance. Examples of input include “GPS: 35.6,139.7 (home), Wi-Fi: home SSID,” “GPS: 34.7,135.5 (travel destination),” and output examples include “recommended acquisition: indoor images and audio,” “recommended acquisition: environmental noise reduction,” “recommended acquisition: add local information.” The reception unit provides acquisition guidance and points to note according to the location information to the parent's device, and sends the acquired data to the preprocessing and analysis units. Subsequent processing involves using location-tagged data for health condition evaluation and environmental factor analysis in AI analysis. Technical effects include: (1) improved AI analysis accuracy through acquisition of highly relevant data based on geographic location; (2) realization of health condition evaluation considering environmental factors; (3) reduced burden on parents and improved data acquisition efficiency; (4) realization of location-linked data acquisition, unlike conventional uniform acquisition methods. Specific application fields include home health management AI, remote diagnostic support, health observation during travel or business trips, health monitoring in infectious disease outbreak areas, and environment-linked health management.

The reception unit can analyze a parent's social media activity and acquire related information when acquiring images and audio. The reception unit analyzes a parent's social media activity and acquires related information when acquiring images and audio. For example, if the parent posts about the child's health on social media, the system can refer to that content during acquisition. If the parent mentions specific symptoms on social media, the system can acquire information related to those symptoms. Furthermore, if the parent exchanges information with other parents on social media, the system can refer to that content during acquisition. To analyze a parent's social media activity, for example, post content and follower information can be used. As a result, the reception unit can analyze a parent's social media activity and acquire related information. For example, by referring to the parent's posts about the child's health on social media, the system can accurately evaluate the child's health condition. This allows parents to efficiently submit images and audio and receive analysis and evaluation by AI. Specifically, with the parent's consent, the reception unit obtains data such as post history, comments, like history, and follower attributes from major social media APIs (e.g., health-related SNS, microblogs, community bulletin boards, etc.). The social media analysis module uses natural language processing models (e.g., BERT-based text classifiers, topic modeling) to extract health-related keywords (e.g., “cough,” “fever,” “hospital,” “anxiety,” etc.), symptom categories, and parent interest/anxiety scores from post content. Examples of input include “Post: ‘I'm worried because my child is coughing,’” “Comment: ‘Many children have the same symptoms,’” and output examples include “related symptoms: cough, anxiety score: 0.8,” “recommended information to acquire: breathing sound, cough recording.” Based on these analysis results, the reception unit prioritizes acquisition of highly relevant information (e.g., cough recording, close-up image of complexion) as guidance during image and audio acquisition. Furthermore, if the parent is exchanging information with other parents, the system automatically proposes useful acquisition methods and points to note shared in the community. Subsequent processing involves using social media-derived information as supplementary information for health condition evaluation and symptom estimation in the AI analysis unit. Technical effects include: (1) improved AI analysis accuracy through acquisition of related data based on the parent's actual interests, anxieties, and symptom information; (2) reduced psychological burden on parents and increased system usage rate; (3) realization of social-linked data acquisition, unlike conventional uniform acquisition instructions; (4) advanced health management support through utilization of community knowledge. Specific application fields include home health management AI, remote diagnostic support, health consultation chatbots, community-linked health observation, and child-rearing support SNS-linked health management.

The analysis unit can estimate a parent's emotion and adjust the expression method of analysis based on the estimated emotion. The analysis unit estimates a parent's emotion and adjusts the expression method of analysis based on the estimated emotion. To estimate a parent's emotion, for example, facial expression recognition or audio analysis technology can be used. For example, if the parent is anxious, the analysis result can be displayed simply. If the parent is relaxed, detailed analysis results can be displayed. Furthermore, if the parent is busy, the analysis result can be displayed with key points highlighted. As a result, the analysis unit can adjust the expression method of analysis based on the parent's emotion. For example, if the parent is anxious, displaying simple and easy-to-understand analysis results can reduce the parent's anxiety. This allows parents to check their child's health condition with peace of mind. Specifically, the analysis unit receives facial images (e.g., front camera face images, resolution 640×480 pixels, 8-bit depth) and audio data (e.g., 16 kHz sampling, 16-bit PCM) obtained from the parent's device as input. The analysis unit uses a facial expression recognition module (e.g., convolutional neural network-based facial expression classifier) to estimate emotion labels and scores (e.g., 0.0-1.0) such as “anxious,” “relaxed,” or “busy” from the parent's facial image. The audio analysis module extracts features such as MFCC, pitch, speech rate, and voice intensity from the parent's speech audio and estimates emotion using recurrent neural networks or Transformer-based models. Examples of input include “parent's facial image (expression: furrowed brow, downturned mouth corners)” and “parent's audio (high pitch, fast speech rate),” and output examples include “emotion label: anxious,” “emotion score: 0.85.” Based on the estimated emotion label and score, the analysis unit's analysis result expression control module automatically adjusts the level of detail and style of the displayed content. For example, rules or machine learning-based expression control algorithms are applied, such as “if emotion score is 0.8 or higher and anxious, display only key points simply,” “if emotion score is less than 0.3 and relaxed, display detailed analysis content and supporting data,” “if emotion score is 0.5 or higher and busy, display key points as bullet points.” Examples of analysis result output include “health condition: caution, reason: complexion is pale,” “health condition: normal, details: complexion score 0.8, breathing abnormality 0.1.” Subsequent processing involves sending the adjusted analysis results to the evaluation unit and summarization unit for feedback to parents and medical institutions. Technical effects include: (1) improved quality of information transmission through optimal display of analysis results according to the parent's psychological state; (2) reduced anxiety and improved understanding for parents; (3) improved reliability and satisfaction with AI analysis; (4) realization of personalized interaction, unlike conventional uniform analysis result display. Specific application fields include home health management AI, remote diagnostic support, child monitoring IoT, mental health care support, and stress detection-based health management in care settings.

The analysis unit can adjust the level of detail of analysis based on the importance of images and audio during analysis. The analysis unit adjusts the level of detail of analysis based on the importance of images and audio during analysis. To evaluate the importance of images and audio, for example, the urgency of the content or the value of the information can be considered. For example, images and audio with high importance can be analyzed in detail, while those with low importance can be analyzed briefly. The priority of analysis can also be determined according to importance. As a result, the analysis unit can adjust the level of detail of analysis based on the importance of images and audio. For example, by analyzing images and audio with high importance in detail, the system can accurately evaluate the child's health condition. This enables the AI to efficiently perform analysis and quickly provide evaluation results. Specifically, the analysis unit first performs preprocessing such as noise removal and feature extraction on image data (e.g., RGB images, resolution 1920×1080 pixels) and audio data (e.g., 16 kHz sampling, 16-bit PCM) received from the reception unit. Next, the importance evaluation module (e.g., image abnormality estimator using convolutional neural networks, audio abnormality scorer) calculates urgency scores (e.g., 0.0-1.0) and information value scores (e.g., 0.0-1.0) for each data. Examples of input include “image: pale complexion, audio: severe cough,” “image: normal, audio: normal,” and output examples include “importance score: 0.9 (high), 0.2 (low).” The analysis unit automatically selects either the detailed analysis module (e.g., multilayer CNN for detailed feature extraction, LSTM for time-series analysis) or the simple analysis module (e.g., single-layer CNN, simple threshold judgment) according to the importance score. For example, “if importance score is 0.7 or higher, perform detailed analysis,” “if less than 0.7, perform simple analysis.” In detailed analysis, multiple features such as complexion, facial expressions, body movements, breathing sounds, and abnormal sounds are extracted as vectors of 128 dimensions or more, and abnormality scores and symptom labels are output with high accuracy. In simple analysis, only major features are extracted, and binary judgment of abnormality or simple scores are output. Subsequent processing involves sending analysis results according to the level of detail to the evaluation unit for health condition determination and medical service desk connection. Technical effects include: (1) improved computational efficiency through optimal allocation of AI resources; (2) reduced risk of misjudgment through high-precision analysis of important data; (3) improved overall throughput through rapid processing of low-importance data; (4) realization of dynamic detail control, unlike conventional uniform analysis procedures. Specific application fields include home health management AI, emergency triage, remote diagnostic support, medical image/audio analysis systems, and IoT health observation devices.

The analysis unit can apply different analysis algorithms according to the category of images and audio during analysis. The analysis unit applies different analysis algorithms according to the category of images and audio during analysis. Categories of images and audio may include, for example, medical images or everyday audio. For example, a specific algorithm may be used for complexion analysis, and a different algorithm may be used for breathing analysis. Furthermore, another algorithm may be used for body movement analysis. The analysis unit can apply the optimal analysis algorithm according to these categories. As a result, the analysis unit can apply different analysis algorithms according to the category of images and audio. For example, by using a face recognition algorithm for complexion analysis and a speech pattern recognition algorithm for breathing analysis, the system can accurately evaluate the child's health condition. This enables the AI to efficiently perform analysis and quickly provide evaluation results. Specifically, the analysis unit first uses a category classification module (e.g., image content classification CNN, audio content classifier) to automatically assign category labels such as “complexion,” “facial expression,” “body movement,” “breathing sound,” “cough,” or “environmental sound” to image and audio data received from the reception unit. Examples of input include “image: complexion,” “audio: breathing sound,” “image: body movement,” “audio: cough,” and output examples include “category: complexion,” “category: breathing sound.” The analysis unit automatically selects and applies the optimal analysis algorithm for each category (e.g., CNN with color space conversion for complexion analysis, multiclass Softmax for facial expression classification, 3D-CNN for body movement analysis, LSTM for breathing sound analysis, spectral pattern matching for cough detection). For example, for the “complexion” category, Lab color space conversion, face region extraction, and CNN-based color feature extraction are applied; for the “breathing sound” category, MFCC extraction and LSTM-based time-series anomaly detection are applied; for the “body movement” category, posture estimation algorithms and time-series image analysis are applied. Examples of analysis result output include “complexion score: 0.2,” “breathing abnormality: 0.85,” “body movement abnormality: 0.7.” Subsequent processing involves integrating category-specific analysis results for use in health condition determination and urgency evaluation in the evaluation unit. Technical effects include: (1) improved analysis accuracy through application of algorithms optimized for data content; (2) improved adaptability to diverse symptom patterns through simultaneous processing of multiple categories; (3) efficient use of computational resources; (4) realization of flexible analysis flow, unlike conventional uniform algorithm application. Specific application fields include home health management AI, remote diagnostic support, medical image/audio analysis, IoT health observation devices, and symptom-specific AI diagnostic support.

The analysis unit can estimate a parent's emotion and adjust the length of analysis based on the estimated emotion. The analysis unit estimates a parent's emotion and adjusts the length of analysis based on the estimated emotion. To estimate a parent's emotion, for example, facial expression recognition or audio analysis technology can be used. For example, if the parent is anxious, the analysis can be short and focused on key points. If the parent is relaxed, the analysis can be detailed. Furthermore, if the parent is busy, the analysis can be performed quickly. As a result, the analysis unit can adjust the length of analysis based on the parent's emotion. For example, if the parent is anxious, performing a short and focused analysis can reduce the parent's anxiety. This allows parents to check their child's health condition with peace of mind. Specifically, the analysis unit receives the parent's facial image and audio data as input, and uses facial expression recognition modules and audio emotion estimation modules (e.g., CNN+Softmax classifier, LSTM-based emotion estimator) to estimate emotion labels and scores. Examples of input include “parent's facial image (expression: anxious),” “parent's audio (fast speech rate),” and output examples include “emotion label: anxious, score: 0.9.” Based on the estimated emotion score, the analysis result generation module automatically adjusts the length and level of detail of the output text. For example, rules are applied such as “if emotion score is 0.8 or higher and anxious, display analysis results in two sentences or less with only key points,” “if emotion score is less than 0.3 and relaxed, display detailed analysis content and supporting data in five sentences or more,” “if emotion score is 0.5 or higher and busy, display only key points in one sentence.” Examples of analysis result output include “health condition: caution,” “health condition: normal, details: complexion score 0.8, breathing abnormality 0.1, body movement abnormality 0.2.” Subsequent processing involves sending the adjusted analysis results to the evaluation unit and summarization unit for feedback to parents and medical institutions. Technical effects include: (1) optimization of information transmission through control of analysis result length according to the parent's psychological state; (2) reduced anxiety and improved understanding for parents; (3) improved reliability and satisfaction with AI analysis; (4) realization of personalized interaction, unlike conventional uniform analysis result length. Specific application fields include home health management AI, remote diagnostic support, child monitoring IoT, mental health care support, and stress detection-based health management in care settings.

The analysis unit can determine the priority of analysis based on the submission timing of images and audio during analysis. The analysis unit determines the priority of analysis based on the submission timing of images and audio during analysis. To evaluate the submission timing of images and audio, for example, the submission date and frequency may be considered. For instance, images and audio submitted recently may be analyzed preferentially, while those with older submission dates may be processed later. Additionally, the order of analysis can be adjusted according to the submission timing. Thus, the analysis unit can determine the priority of analysis based on the submission timing of images and audio. For example, by preferentially analyzing recently submitted images and audio, the health condition of a child can be evaluated promptly. As a result, the AI can efficiently perform analysis and provide evaluation results quickly. Specifically, the analysis unit receives as input the submission timestamp (e.g., UNIX time, submission date string) and submission frequency information attached to image and audio data received from the reception unit. The submission timing evaluation module compares the submission dates of each data and automatically assigns a high priority score (e.g., 1.0) to the latest data and a low priority score (e.g., 0.1) to older data. Example inputs include “Image A: 2024-06-01 20:00, Image B: 2024-05-28 18:00”, and example outputs include “Priority: Image A>Image B”. The analysis unit automatically adjusts the order of the analysis queue based on the priority score and performs detailed analysis sequentially from the latest data. In subsequent processing, the analysis results of high-priority data are quickly sent to the evaluation unit and summarization unit, optimizing emergency response and feedback to parents. Technical effects include: (1) improved responsiveness to changes in health condition by prioritizing the latest data; (2) efficient allocation of analysis resources; (3) time-series optimization differing from conventional uniform analysis order; (4) rapid information provision to parents and medical institutions. Specific application fields include home health management AI, emergency triage, remote diagnostic support, medical image and audio analysis systems, and IoT health monitoring terminals.

The analysis unit can adjust the order of analysis based on the relevance between images and audio during analysis. The analysis unit adjusts the order of analysis based on the relevance between images and audio during analysis. To evaluate the relevance between images and audio, for example, the degree of content matching and related topics may be considered. For instance, highly relevant images and audio may be analyzed preferentially, while those with low relevance may be processed later. Additionally, the order of analysis can be adjusted according to relevance. Thus, the analysis unit can adjust the order of analysis based on the relevance between images and audio. For example, by preferentially analyzing highly relevant images and audio, the health condition of a child can be evaluated accurately. As a result, the AI can efficiently perform analysis and provide evaluation results quickly. Specifically, the analysis unit uses a relevance evaluation module (e.g., cosine similarity calculation between image and audio feature vectors, topic modeling) to calculate a content matching score (e.g., 0.0-1.0) for each image and audio received from the reception unit. Example inputs include “Image A: pale complexion, Audio A: rough breathing sound”, “Image B: normal, Audio B: normal”, and example outputs include “Relevance score: Image A-Audio A=0.95, Image B-Audio B=0.2”. The analysis unit prioritizes analysis of pairs with high relevance scores and controls the analysis queue so that pairs with low scores are processed later. Furthermore, the level of detail of the analysis algorithm and whether to perform integrated processing are automatically adjusted according to relevance. In subsequent processing, the analysis results of highly relevant data are sent to the evaluation unit and summarization unit, contributing to improved accuracy in health condition determination and urgency assessment. Technical effects include: (1) reduced risk of misjudgment through integrated analysis based on content matching between images and audio; (2) improved accuracy in health condition estimation by prioritizing highly relevant data; (3) efficient allocation of analysis resources; (4) realization of a content-linked analysis flow differing from conventional uniform analysis order. Specific application fields include home health management AI, remote diagnostic support, medical image and audio analysis, IoT health monitoring terminals, and symptom-specific AI diagnostic support.

The evaluation unit can estimate a parent's emotion and adjust the evaluation criteria based on the estimated emotion. The evaluation unit estimates a parent's emotion and adjusts the evaluation criteria based on the estimated emotion. To estimate a parent's emotion, for example, facial recognition and audio analysis techniques may be used. For instance, if the parent is feeling anxious, the evaluation can be performed using stricter criteria. If the parent is relaxed, standard criteria can be applied. Furthermore, if the parent is busy, the evaluation can be performed quickly. Thus, the evaluation unit can adjust the evaluation criteria based on the parent's emotion. For example, by evaluating with stricter criteria when the parent is anxious, the parent's anxiety can be alleviated. As a result, the parent can check the child's health condition with peace of mind. Specifically, the evaluation unit receives facial images (e.g., face images from the front camera, resolution 640×480 pixels, 8-bit depth) and audio data (e.g., 16 kHz sampling, 16-bit PCM) obtained from the parent's device as input. The evaluation unit uses a facial recognition module (convolutional neural network-based facial classifier) and an audio emotion estimation module (recurrent neural network or Transformer-based model) to estimate the parent's emotion label (e.g., “anxious”, “relaxed”, “busy”) and emotion score (0.0-1.0). Example inputs include “parent's face image (expression: furrowed brow, downturned mouth corners)”, “parent's audio (high pitch, fast speaking rate)”, and example outputs include “emotion label: anxious”, “emotion score: 0.85”. Based on the estimated emotion label and score, the evaluation criteria adjustment module automatically changes the threshold and weight parameters for health condition determination. For example, “if the emotion score is 0.8 or higher and anxious, the threshold for complexion score and breathing abnormality is made stricter, making emergency judgment more likely than usual”; “if the emotion score is less than 0.3 and relaxed, standard evaluation criteria are applied”; “if the emotion score is 0.5 or higher and busy, only major items are evaluated quickly”; such rule-based or machine learning-based criteria adjustment algorithms are applied. The evaluation unit integrates multidimensional feature vectors (e.g., complexion score, breathing abnormality, expression label, body movement abnormality) received from the image analysis unit and audio analysis unit based on the adjusted evaluation criteria, and outputs a health condition label (e.g., “emergency”, “caution”, “normal”) and urgency score (0.0-1.0). Example outputs include “emergency”, “caution”, “normal”, or “urgency score: 0.92”. In subsequent processing, the evaluation results are sent to the connection unit and summarization unit and used for medical service desk connection and natural language summary generation. Technical effects include: (1) improved sense of security and reliability through dynamic optimization of evaluation criteria according to the parent's psychological state; (2) realization of personalized health condition evaluation by AI; (3) reduced risk of misjudgment through a flexible judgment flow differing from conventional uniform evaluation criteria; (4) alleviation of parental anxiety and improvement of satisfaction. Specific application fields include home health management AI, remote diagnostic support, child monitoring IoT, mental health care support, and stress detection-based health management in nursing care settings.

The evaluation unit can improve the accuracy of evaluation by considering the interrelationship between images and audio during evaluation. The evaluation unit improves the accuracy of evaluation by considering the interrelationship between images and audio during evaluation. To evaluate the interrelationship between images and audio, for example, the degree of content matching and related topics may be considered. For instance, the degree of matching between images and audio can be reflected in the evaluation, and the interrelationship between images and audio can be analyzed. Additionally, the accuracy of evaluation can be improved based on the interrelationship between images and audio. Thus, the evaluation unit can improve the accuracy of evaluation by considering the interrelationship between images and audio. For example, by reflecting the degree of matching between images and audio in the evaluation, the health condition of a child can be evaluated accurately. As a result, the AI can efficiently perform evaluation and provide evaluation results quickly. Specifically, the evaluation unit receives multidimensional feature vectors (e.g., complexion score, expression label, body movement abnormality, breathing abnormality score, abnormal sound label, etc.) from the image analysis unit and audio analysis unit as input. The evaluation unit uses a relevance evaluation module (e.g., cosine similarity calculation between feature vectors, topic modeling, cross-modal attention mechanism) to calculate a content matching score (e.g., 0.0-1.0) for each image and audio. Example inputs include “Image A: pale complexion, Audio A: rough breathing sound”, “Image B: normal, Audio B: normal”, and example outputs include “Relevance score: Image A-Audio A=0.95, Image B-Audio B=0.2”. When the relevance score is high, the evaluation unit integrates the information from images and audio and increases the weight for health condition determination; when the score is low, the reliability of individual judgments is decreased, and the parameters of the evaluation algorithm are dynamically adjusted. For example, “if the complexion score is low and the breathing abnormality is high, and the relevance score is 0.8 or higher, the threshold for emergency judgment is lowered”; “if the relevance score is less than 0.3, additional data acquisition is prompted”; such rules are applied. The evaluation unit outputs the integrated evaluation result as a health condition label (e.g., “emergency”, “caution”, “normal”) and urgency score (e.g., 0.92), and sends it to the subsequent connection unit and summarization unit. In subsequent processing, the evaluation results are used for medical service desk connection and natural language summary generation. Technical effects include: (1) reduced risk of misjudgment through integrated evaluation based on content matching between images and audio; (2) improved accuracy in health condition estimation through mutual complementation of multiple modality information; (3) automatic optimization of flexible evaluation criteria by AI; (4) realization of highly accurate health management support differing from conventional single modality evaluation. Specific application fields include home health management AI, remote diagnostic support, medical image and audio analysis, IoT health monitoring terminals, and symptom-specific AI diagnostic support.

The evaluation unit can perform evaluation by considering the attribute information of the submitter of images and audio during evaluation. The evaluation unit performs evaluation by considering the attribute information of the submitter of images and audio during evaluation. Attribute information of the submitter includes, for example, age, gender, occupation, etc. For instance, evaluation can be performed by considering the age and gender of the submitter. Additionally, evaluation can be performed by considering the health condition of the submitter. Furthermore, evaluation can be performed by considering the submitter's past submission history. Thus, the evaluation unit can perform evaluation by considering the attribute information of the submitter. For example, by considering the age and gender of the submitter, the health condition of a child can be evaluated accurately. As a result, the AI can efficiently perform evaluation and provide evaluation results quickly. Specifically, the evaluation unit receives attribute information attached to image and audio data received from the reception unit (e.g., age, gender, occupation, medical history, allergy information, past health checkup results, submission history database) as input. The evaluation unit uses an attribute information analysis module (e.g., attribute vector encoder, decision tree-based attribute weighting device) to automatically adjust the optimal evaluation criteria and scoring parameters for each submitter. Example inputs include “age: 5 years, gender: male, medical history: asthma”, “age: 10 years, gender: female, medical history: none”, and example outputs include “evaluation criteria: breathing abnormality threshold 0.6 (asthmatic child), 0.8 (healthy child)”. The evaluation unit also refers to past submission history (e.g., health condition over the past 10 submissions, frequency of abnormal detection, physician diagnosis results) and performs individually optimized health condition determination. For example, “if there is a history of respiratory disease, increase the weight for breathing abnormality”; “if the abnormal detection rate is high in past submissions, make the threshold stricter”; such rules are applied. The evaluation result is output as a health condition label or urgency score and sent to the connection unit and summarization unit. In subsequent processing, the individualized evaluation result is used for medical service desk connection and natural language summary generation. Technical effects include: (1) improved accuracy through personalized health condition evaluation according to submitter attributes; (2) reduced risk of misjudgment by utilizing medical history and history information; (3) automatic optimization of flexible evaluation criteria by AI; (4) realization of individually optimized health management support differing from conventional uniform evaluation methods. Specific application fields include home health management AI, remote diagnostic support, medical image and audio analysis, IoT health monitoring terminals, and personalized medical support.

The evaluation unit can estimate a parent's emotion and adjust the order of displaying evaluation results based on the estimated emotion. The evaluation unit estimates a parent's emotion and adjusts the order of displaying evaluation results based on the estimated emotion. To estimate a parent's emotion, for example, facial recognition and audio analysis techniques may be used. For instance, if the parent is feeling anxious, important results can be displayed first. If the parent is relaxed, detailed results can be displayed sequentially. Furthermore, if the parent is busy, key results can be displayed first. Thus, the evaluation unit can adjust the order of displaying evaluation results based on the parent's emotion. For example, by displaying important results first when the parent is anxious, the parent's anxiety can be alleviated. As a result, the parent can check the child's health condition with peace of mind. Specifically, the evaluation unit receives the parent's facial images and audio data as input, and estimates emotion labels and scores using a facial recognition module and audio emotion estimation module (e.g., CNN+Softmax classifier, LSTM-based emotion estimator). Example inputs include “parent's face image (expression: anxious)”, “parent's audio (fast speaking rate)”, and example outputs include “emotion label: anxious, score: 0.9”. Based on the estimated emotion score, the evaluation result display order control module automatically adjusts the priority and order of displayed content. For example, “if the emotion score is 0.8 or higher and anxious, display emergency judgment and abnormal detection results first”; “if the emotion score is less than 0.3 and relaxed, display detailed analysis content and supporting data sequentially”; “if the emotion score is 0.5 or higher and busy, display only key points first in bullet points”; such rules are applied. Example outputs of evaluation results include “Health condition: emergency, reason: pale complexion”, “Health condition: normal, details: complexion score 0.8, breathing abnormality 0.1”, etc. In subsequent processing, the adjusted evaluation results are sent to the parent's device or medical institution, contributing to rapid decision-making and improved sense of security. Technical effects include: (1) improved quality of information transmission through optimal display of evaluation results according to the parent's psychological state; (2) alleviation of parental anxiety and improved understanding; (3) improved reliability and satisfaction of AI evaluation; (4) realization of personalized interaction differing from conventional uniform result display. Specific application fields include home health management AI, remote diagnostic support, child monitoring IoT, mental health care support, and stress detection-based health management in nursing care settings.

The evaluation unit can perform evaluation by considering the geographic distribution of images and audio during evaluation. The evaluation unit performs evaluation by considering the geographic distribution of images and audio during evaluation. To evaluate the geographic distribution of images and audio, for example, regional distribution and geographic bias may be considered. For instance, evaluation can be performed by considering the submitter's place of residence. Additionally, evaluation can be performed by considering the health condition in each region. Furthermore, the accuracy of evaluation can be improved based on geographic distribution. Thus, the evaluation unit can perform evaluation by considering the geographic distribution of images and audio. For example, by considering the submitter's place of residence, the health condition of a child can be evaluated accurately. As a result, the AI can efficiently perform evaluation and provide evaluation results quickly. Specifically, the evaluation unit receives geographic location information (e.g., GPS coordinates, address, region code) attached to image and audio data received from the reception unit as input. The evaluation unit uses a geographic distribution analysis module (e.g., clustering algorithm, geographic information system integration) to automatically extract health condition trends and epidemic information for each region. Example inputs include “GPS: 35.6,139.7 (Tokyo), residence: Osaka”, and example outputs include “Region: Tokyo, epidemic: influenza, evaluation criteria: fever threshold 0.37”. The evaluation unit dynamically adjusts evaluation criteria and judgment thresholds by considering health risks and medical resource status for each region. For example, “in regions with infectious disease outbreaks, fever and cough thresholds are made stricter”; “in regions with limited medical resources, early warning is prioritized”; such rules are applied. The evaluation result is output as a health condition label or urgency score and sent to the connection unit and summarization unit. In subsequent processing, evaluation results based on geographic distribution are used for medical service desk connection and natural language summary generation. Technical effects include: (1) improved accuracy through health condition evaluation reflecting regional characteristics and epidemic status; (2) reduced risk of misjudgment by correcting geographic bias; (3) automatic optimization of flexible evaluation criteria by AI; (4) realization of region-adaptive health management support differing from conventional uniform evaluation methods. Specific application fields include home health management AI, remote diagnostic support, health monitoring in epidemic regions, medical resource allocation support, and IoT health monitoring terminals.

The evaluation unit can improve the accuracy of evaluation by referring to related literature of images and audio during evaluation. The evaluation unit improves the accuracy of evaluation by referring to related literature of images and audio during evaluation. Related literature includes, for example, academic papers and technical reports. For instance, evaluation criteria can be set by referring to related literature. Additionally, the accuracy of evaluation can be improved by referring to related literature. Furthermore, evaluation results can be supplemented based on related literature. Thus, the evaluation unit can improve the accuracy of evaluation by referring to related literature of images and audio. For example, by referring to related literature, the health condition of a child can be evaluated accurately. As a result, the AI can efficiently perform evaluation and provide evaluation results quickly. Specifically, the evaluation unit receives image and audio analysis results (e.g., complexion score, breathing abnormality, abnormal sound label, etc.) as input, and automatically searches and extracts literature information related to the relevant symptoms and analysis results from a related literature database (e.g., PubMed, medical guidelines, technical report collections). The literature search module (e.g., BERT-based literature search engine, topic modeling) is used to calculate similarity scores (e.g., 0.0-1.0) between analysis results and literature content, as well as recommended evaluation criteria. Example inputs include “symptom: cough, complexion: pale, breathing abnormality: 0.85”, and example outputs include “recommended evaluation criteria: emergency judgment if breathing abnormality is 0.7 or higher (literature ID: 12345)”. The evaluation unit reflects evaluation criteria and supplementary information based on literature in the health condition determination algorithm, and attaches judgment grounds and reference literature information to the evaluation results. For example, outputs such as “emergency judgment: complexion score 0.2, breathing abnormality 0.9 (reference literature ID: 12345)” are possible. In subsequent processing, evaluation results with literature references are used for medical service desk connection and natural language summary generation, contributing to accountability and improved reliability for physicians and parents. Technical effects include: (1) improved accuracy through health condition evaluation based on the latest medical knowledge and evidence; (2) improved explainability and reliability by clarifying AI judgment grounds; (3) realization of evidence-based evaluation differing from conventional empirical or black-box judgments; (4) diagnostic support and educational use in medical settings. Specific application fields include home health management AI, remote diagnostic support, medical image and audio analysis, medical education support, and evidence-based diagnostic support.

The connection unit can estimate a parent's emotion and adjust the connection method based on the estimated emotion. The connection unit estimates a parent's emotion and adjusts the connection method based on the estimated emotion. To estimate a parent's emotion, for example, facial recognition and audio analysis techniques may be used. For instance, if the parent is feeling anxious, the system can quickly connect to a dedicated service desk. If the parent is relaxed, the connection can proceed according to the usual procedure. Furthermore, if the parent is busy, a reminder can be set to connect later. Thus, the connection unit can adjust the connection method based on the parent's emotion. For example, by quickly connecting to a dedicated service desk when the parent is anxious, the parent's anxiety can be alleviated. As a result, the parent can check the child's health condition with peace of mind. Specifically, the connection unit receives facial images (e.g., face images from the front camera, resolution 640×480 pixels, 8-bit depth) and audio data (e.g., 16 kHz sampling, 16-bit PCM) obtained from the parent's device as input. The connection unit uses a facial recognition module (convolutional neural network-based facial classifier) and an audio emotion estimation module (recurrent neural network or Transformer-based model) to estimate the parent's emotion label (e.g., “anxious”, “relaxed”, “busy”) and emotion score (0.0-1.0). Example inputs include “parent's face image (expression: furrowed brow, downturned mouth corners)”, “parent's audio (high pitch, fast speaking rate)”, and example outputs include “emotion label: anxious”, “emotion score: 0.85”. Based on the estimated emotion label and score, the connection method control module automatically adjusts the connection procedure, destination selection, and connection timing. For example, “if the emotion score is 0.8 or higher and anxious, immediate connection to the emergency consultation desk is prioritized”; “if the emotion score is less than 0.3 and relaxed, connect to the general consultation desk according to the usual procedure”; “if the emotion score is 0.5 or higher and busy, prioritize setting a reminder and propose connecting later”; such rule-based or machine learning-based connection method determination algorithms are applied. The connection unit automatically executes procedures such as API calls, session generation, authentication information assignment, and communication channel establishment according to the selected connection method, and immediately sends connection completion notifications or connection links to the parent's device. In subsequent processing, connection history and changes in the parent's emotion are recorded and used for optimizing future connections and strengthening collaboration with medical institutions. Technical effects include: (1) accelerated and optimized medical access through connection method control according to the parent's psychological state; (2) realization of personalized connection experiences by AI; (3) improved sense of security and satisfaction for parents through flexible connection flows differing from conventional uniform connection procedures; (4) improved connection reliability and reduced risk of misconnection. Specific application fields include home health management AI, remote diagnostic support, emergency triage, stress detection-based medical access support, and emotion-linked medical collaboration in nursing care settings.

The connection unit can adjust the level of detail of connection based on the importance of evaluation results at the time of connection. The connection unit adjusts the level of detail of connection based on the importance of evaluation results at the time of connection. To evaluate the importance of evaluation results, for example, the urgency of the content and the value of the information can be considered. For instance, evaluation results with high importance can be connected in detail, while those with low importance can be connected in a simplified manner. Additionally, the priority of connection can be determined according to the importance. Thus, the connection unit can adjust the level of detail of connection based on the importance of evaluation results. For example, by connecting highly important evaluation results in detail, the child's health condition can be accurately evaluated. As a result, the AI can efficiently perform connections and promptly provide evaluation results. Specifically, the connection unit receives as input health condition labels (e.g., “Emergency,” “Caution,” “Normal”), urgency scores (e.g., continuous values from 0.0 to 1.0), and importance scores (e.g., 0.0 to 1.0) received from the evaluation unit. The connection unit quantifies the urgency and information value of each evaluation result using an importance evaluation module (e.g., decision tree-based importance classifier, neural network scoring). Examples of input include “Health condition: Emergency, Urgency score: 0.92, Importance score: 0.95” and “Health condition: Normal, Urgency score: 0.2, Importance score: 0.3”; examples of output include “Connection detail level: High (video call+detailed information transfer)” and “Connection detail level: Low (simple chat notification).” The connection unit automatically selects either a detailed connection module (e.g., real-time video call connection to medical institutions, detailed health data transfer, immediate alert transmission to doctors) or a simplified connection module (e.g., chat notification, setting a follow-up reminder) according to the importance score. For example, rules such as “detailed connection if importance score is 0.8 or higher” and “simplified connection if less than 0.8” are applied. Subsequent processing includes information transfer and medical service desk coordination according to the connection detail level, optimizing notification content and response procedures for parents and medical institutions. Technical effects include: (1) computational efficiency through optimal allocation of connection resources according to the importance of evaluation results; (2) reduction of erroneous responses by high-precision, high-reliability connection of important data; (3) improvement of overall throughput by rapid processing of low-importance data; and (4) dynamic detail control differing from conventional uniform connection procedures. Specific application fields include home health management AI, emergency triage, remote diagnostic support, automatic patient sorting systems for medical institutions, and IoT health monitoring devices.

The connection unit can apply different connection algorithms according to the category of evaluation results at the time of connection. The connection unit applies different connection algorithms according to the category of evaluation results at the time of connection. Categories of evaluation results may include, for example, medical evaluation and psychological evaluation. For instance, a rapid connection algorithm can be applied to evaluation results with high urgency, and a standard connection algorithm can be applied to normal evaluation results. Furthermore, a delayed connection algorithm can be applied to evaluation results with low urgency. The connection unit can apply the optimal connection algorithm according to these categories. Thus, the connection unit can apply different connection algorithms according to the category of evaluation results. For example, by applying a rapid connection algorithm to evaluation results with high urgency, the child's health condition can be evaluated quickly. As a result, the AI can efficiently perform connections and promptly provide evaluation results. Specifically, the connection unit receives as input category labels (e.g., “medical evaluation,” “psychological evaluation,” “lifestyle guidance,” etc.) and urgency scores attached to evaluation results received from the evaluation unit. A category classification module (e.g., multi-class Softmax classifier, rule-based classifier) automatically selects the optimal connection algorithm for each evaluation result (e.g., immediate API call, standard queuing, delayed batch processing). Examples of input include “Category: medical evaluation, urgency: high” and “Category: psychological evaluation, urgency: low”; examples of output include “Connection algorithm: immediate connection” and “Connection algorithm: delayed connection.” The connection unit executes different connection procedures for each category (e.g., medical evaluation uses video call API to medical institutions, psychological evaluation uses chat connection to counselors, lifestyle guidance uses automatic advice notification). Furthermore, the connection destination and information transfer content are dynamically adjusted according to urgency and category. Subsequent processing includes recording connection history and response results, which are used for optimizing future connections and strengthening collaboration with medical and psychological support institutions. Technical effects include: (1) improved response accuracy by applying connection algorithms optimized for evaluation categories; (2) enhanced adaptability to diverse health support patterns by simultaneous processing of multiple categories; (3) efficient use of computational resources; and (4) realization of flexible connection flows differing from conventional uniform connection procedures. Specific application fields include home health management AI, remote diagnostic support, medical/psychological/lifestyle support collaboration systems, and IoT health monitoring devices.

The connection unit can estimate a parent's emotion and determine the priority of connection based on the estimated emotion. The connection unit estimates a parent's emotion and determines the priority of connection based on the estimated emotion. To estimate a parent's emotion, for example, facial recognition and audio analysis techniques can be used. For instance, if the parent is feeling anxious, connection can be made promptly. If the parent is relaxed, connection can be made using the normal procedure. Furthermore, if the parent is busy, a reminder can be set to connect later. Thus, the connection unit can determine the priority of connection based on the parent's emotion. For example, by connecting promptly when the parent is feeling anxious, the parent's anxiety can be alleviated. As a result, the parent can check the child's health condition with peace of mind. Specifically, the connection unit receives as input facial images and audio data obtained from the parent's terminal, and estimates emotion labels and scores (e.g., “anxiety” 0.9, “relaxed” 0.2, “busy”0.7, etc.) using a facial recognition module (convolutional neural network) and an audio emotion estimation module (recurrent neural network or Transformer-type model). Examples of input include “parent's facial image (expression: anxiety)” and “parent's audio (fast speech rate)”; examples of output include “emotion label: anxiety, score: 0.9.” The connection unit automatically adjusts the order and immediacy of the connection queue using a connection priority determination module based on the estimated emotion score. For example, rules such as “immediate connection is prioritized if the emotion score is 0.8 or higher and anxiety is detected,” “normal priority if the emotion score is less than 0.3 and relaxed is detected,” and “reminder setting is prioritized if the emotion score is 0.5 or higher and busy is detected” are applied. The connection unit immediately notifies the parent's terminal of connection instructions and guidance according to the priority, and records connection history and emotion transitions. Subsequent processing includes rapid transfer of high-priority connections to medical institutions or consultation desks, contributing to the parent's sense of security and optimization of medical access. Technical effects include: (1) improved emergency response capability by controlling connection priority according to the parent's psychological state; (2) realization of personalized connection experiences by AI; (3) flexible priority control differing from conventional uniform connection order; and (4) improved sense of security and satisfaction for parents. Specific application fields include home health management AI, remote diagnostic support, emergency response-type health monitoring, stress detection-type medical access support, and priority control-type medical collaboration in care settings.

The connection unit can adjust the order of connection based on the submission timing of evaluation results at the time of connection. The connection unit adjusts the order of connection based on the submission timing of evaluation results at the time of connection. To evaluate the submission timing of evaluation results, for example, the submission date and time and submission frequency can be considered. For instance, recently submitted evaluation results can be connected preferentially, while older evaluation results can be processed later. Additionally, the order of connection can be adjusted according to the submission timing. Thus, the connection unit can adjust the order of connection based on the submission timing of evaluation results. For example, by preferentially connecting recently submitted evaluation results, the child's health condition can be evaluated quickly. As a result, the AI can efficiently perform connections and promptly provide evaluation results. Specifically, the connection unit receives as input submission timestamps (e.g., UNIX time, submission date and time string) and submission frequency information attached to evaluation results received from the evaluation unit. The submission timing evaluation module compares the submission dates and times of each evaluation result and automatically assigns a high priority score (e.g., 1.0) to the latest data and a low priority score (e.g., 0.1) to older data. Examples of input include “Evaluation A: 2024-06-01 20:00, Evaluation B: 2024-05-28 18:00”; examples of output include “Priority: Evaluation A>Evaluation B.” The connection unit automatically adjusts the order of the connection queue based on the priority score and performs detailed connection in order from the latest data. Subsequent processing includes rapid transfer of high-priority evaluation results to medical institutions or consultation desks, optimizing emergency response and feedback to parents. Technical effects include: (1) improved responsiveness to changes in health condition by prioritizing the latest data; (2) efficient allocation of connection resources; (3) time-series optimization differing from conventional uniform connection order; and (4) rapid information provision to parents and medical institutions. Specific application fields include home health management AI, emergency triage, remote diagnostic support, medical image/audio analysis systems, and IoT health monitoring devices.

The connection unit can adjust the order of connection based on the relevance of evaluation results at the time of connection. The connection unit adjusts the order of connection based on the relevance of evaluation results at the time of connection. To evaluate the relevance of evaluation results, for example, the degree of content match and related topics can be considered. For instance, evaluation results with high relevance can be connected preferentially, while those with low relevance can be processed later. Additionally, the order of connection can be adjusted according to relevance. Thus, the connection unit can adjust the order of connection based on the relevance of evaluation results. For example, by preferentially connecting evaluation results with high relevance, the child's health condition can be evaluated quickly. As a result, the AI can efficiently perform connections and promptly provide evaluation results. Specifically, the connection unit uses a relevance evaluation module (e.g., cosine similarity calculation between feature vectors, topic modeling) to calculate a content match score (e.g., 0.0 to 1.0) between multiple evaluation results received from the evaluation unit. Examples of input include “Evaluation A: pale complexion, Evaluation B: rough breathing sound”; examples of output include “Relevance score: Evaluation A-Evaluation B=0.95.” The connection unit implements connection queue control that preferentially connects evaluation results with high relevance and processes those with low relevance later. Furthermore, the level of detail of the connection algorithm and whether to perform integrated processing are automatically adjusted according to relevance. Subsequent processing includes rapid transfer of highly relevant evaluation results to medical institutions or consultation desks, contributing to improved accuracy of health condition determination and urgency evaluation. Technical effects include: (1) reduction of erroneous responses by integrated connection based on content match of evaluation results; (2) improved accuracy of health condition estimation by prioritizing highly relevant data; (3) efficient allocation of connection resources; and (4) realization of content-linked connection flows differing from conventional uniform connection order. Specific application fields include home health management AI, remote diagnostic support, medical image/audio analysis, IoT health monitoring devices, and symptom-specific AI diagnostic support.

The summarization unit can estimate a parent's emotion and adjust the expression method of summarization based on the estimated emotion. The summarization unit estimates a parent's emotion and adjusts the expression method of summarization based on the estimated emotion. To estimate a parent's emotion, for example, facial recognition and audio analysis techniques may be used. For instance, if the parent is feeling anxious, a simple and easy-to-understand summary can be generated. If the parent is relaxed, a detailed summary can be generated. Furthermore, if the parent is busy, a summary focusing on key points can be generated. Thus, the summarization unit can adjust the expression method of summarization based on the parent's emotion. For example, by generating a simple and easy-to-understand summary when the parent is anxious, the parent's anxiety can be alleviated. As a result, the parent can check the child's health condition with peace of mind.

The summarization unit can adjust the level of detail of summarization based on the importance of evaluation results during summarization generation. The summarization unit adjusts the level of detail of summarization based on the importance of evaluation results during summarization generation. To evaluate the importance of evaluation results, for example, the urgency of the content and the value of the information may be considered. For instance, highly important evaluation results can be summarized in detail, while less important evaluation results can be summarized briefly. Additionally, the priority of summarization can be determined according to importance. Thus, the summarization unit can adjust the level of detail of summarization based on the importance of evaluation results. For example, by summarizing highly important evaluation results in detail, the health condition of a child can be evaluated accurately. As a result, the AI can efficiently perform summarization and provide evaluation results quickly.

The summarization unit can apply different summarization algorithms according to the category of evaluation results during summarization generation. The summarization unit applies different summarization algorithms according to the category of evaluation results during summarization generation. Categories of evaluation results include, for example, medical evaluation and psychological evaluation. For instance, a rapid summarization algorithm can be applied to highly urgent evaluation results, while a standard summarization algorithm can be applied to normal evaluation results. Additionally, a delayed summarization algorithm can be applied to low-urgency evaluation results. The summarization unit can apply the optimal summarization algorithm according to these categories. Thus, the summarization unit can apply different summarization algorithms according to the category of evaluation results. For example, by applying a rapid summarization algorithm to highly urgent evaluation results, the health condition of a child can be evaluated quickly. As a result, the AI can efficiently perform summarization and provide evaluation results quickly.

The summarization unit can estimate a parent's emotion and adjust the length of summarization based on the estimated emotion. The summarization unit estimates a parent's emotion and adjusts the length of summarization based on the estimated emotion. To estimate a parent's emotion, for example, facial recognition and audio analysis techniques may be used. For instance, if the parent is feeling anxious, a short summary focusing on key points can be generated. If the parent is relaxed, a detailed summary can be generated. Furthermore, if the parent is busy, a summary can be generated quickly. Thus, the summarization unit can adjust the length of summarization based on the parent's emotion. For example, by generating a short summary focusing on key points when the parent is anxious, the parent's anxiety can be alleviated. As a result, the parent can check the child's health condition with peace of mind.

The summarization unit can determine the priority of summarization based on the submission timing of evaluation results during summarization generation. The summarization unit determines the priority of summarization based on the submission timing of evaluation results during summarization generation. To evaluate the submission timing of evaluation results, for example, the submission date and frequency may be considered. For instance, recently submitted evaluation results may be summarized preferentially, while those with older submission dates may be processed later. Additionally, the order of summarization can be adjusted according to the submission timing. Thus, the summarization unit can determine the priority of summarization based on the submission timing of evaluation results. For example, by preferentially summarizing recently submitted evaluation results, the health condition of a child can be evaluated promptly. As a result, the AI can efficiently perform summarization and provide evaluation results quickly.

The summarization unit can adjust the order of summarization based on the relevance of evaluation results during summarization generation. The summarization unit adjusts the order of summarization based on the relevance of evaluation results during summarization generation. To evaluate the relevance of evaluation results, for example, the degree of content matching and related topics may be considered. For instance, highly relevant evaluation results may be summarized preferentially, while those with low relevance may be processed later. Additionally, the order of summarization can be adjusted according to relevance. Thus, the summarization unit can adjust the order of summarization based on the relevance of evaluation results. For example, by preferentially summarizing highly relevant evaluation results, the health condition of a child can be evaluated promptly. As a result, the AI can efficiently perform summarization and provide evaluation results quickly.

The learning unit can estimate a parent's emotion and select learning data based on the estimated emotion. The learning unit estimates the parent's emotion and selects learning data based on the estimated emotion. For estimating the parent's emotion, for example, facial expression recognition or audio analysis techniques can be used. For instance, if the parent is feeling anxious, the learning unit can prioritize learning data with high urgency. If the parent is relaxed, the learning unit can learn normal data. Furthermore, if the parent is busy, the learning unit can learn data that focuses on key points. In this way, the learning unit can select learning data based on the parent's emotion. For example, by prioritizing learning of highly urgent data when the parent is feeling anxious, the parent's anxiety can be alleviated. As a result, the AI can learn efficiently and provide evaluation results quickly.

The learning unit can optimize a learning algorithm by referring to past learning data during learning. The learning unit optimizes the learning algorithm by referring to past learning data during learning. To optimize the learning algorithm, for example, past learning data can be analyzed to select the optimal learning algorithm. Additionally, the learning algorithm can be improved based on past learning data. Furthermore, by referring to past learning data, the accuracy of learning can be improved. In this way, the learning unit can optimize the learning algorithm by referring to past learning data. For example, by analyzing past learning data, the optimal learning algorithm can be selected and the accuracy of learning can be improved. As a result, the AI can learn efficiently and provide evaluation results quickly.

The learning unit can estimate a parent's emotion and adjust the frequency of learning based on the estimated emotion. The learning unit estimates the parent's emotion and adjusts the frequency of learning based on the estimated emotion. For estimating the parent's emotion, for example, facial expression recognition or audio analysis techniques can be used. For instance, if the parent is feeling anxious, learning can be performed frequently. If the parent is relaxed, learning can be performed at a normal frequency. Furthermore, if the parent is busy, the frequency of learning can be reduced. In this way, the learning unit can adjust the frequency of learning based on the parent's emotion. For example, by performing learning frequently when the parent is feeling anxious, the parent's anxiety can be alleviated. As a result, the AI can learn efficiently and provide evaluation results quickly.

The learning unit can weight learning data based on the submission timing of images and audio during learning. The learning unit weights learning data based on the submission timing of images and audio during learning. For weighting learning data, for example, the submission date and frequency can be considered. For instance, images and audio submitted recently can be emphasized in learning, while images and audio submitted a long time ago can be de-emphasized. Additionally, the weighting of learning data can be adjusted according to the submission timing. In this way, the learning unit can weight learning data based on the submission timing of images and audio. For example, by emphasizing learning of images and audio submitted recently, the child's health condition can be evaluated quickly. As a result, the AI can learn efficiently and provide evaluation results quickly.

The system according to the embodiment is not limited to the examples described above, and various modifications are possible, for example, as follows.

The health check system may further include a notification unit. The notification unit provides a means for a parent to receive information regarding the child's health condition. For example, the parent can receive notifications regarding the child's health condition using a smartphone or tablet. Additionally, the notification unit can prioritize important information for notification based on the priority set by the parent. For instance, in cases of high urgency, notifications can be sent immediately so that the parent can respond quickly. Furthermore, the notification unit can estimate the parent's emotion and adjust the content and timing of notifications based on the estimated emotion. For example, if the parent is feeling anxious, detailed information can be provided to help the parent feel at ease.

The health check system may further include a history management unit. The history management unit manages past health check results and medical diagnosis results so that the parent can refer to them at any time. For example, the parent can check past health check results and grasp changes in the child's health condition. Additionally, the history management unit can analyze trends in the child's health condition based on past data and provide advice to the parent. For instance, if the child's health condition is deteriorating based on past data, a notification can be sent to alert the parent. Furthermore, the history management unit can estimate the parent's emotion and adjust the display method of the history based on the estimated emotion. For example, if the parent is feeling anxious, a simple and easy-to-understand display can be provided to help the parent feel at ease.

The health check system may further include an advice unit. The advice unit provides advice to the parent based on the analysis result. For example, if the child's health condition is deteriorating, the advice unit can propose appropriate response methods to the parent. Additionally, the advice unit can estimate the parent's emotion and adjust the content and expression method of advice based on the estimated emotion. For instance, if the parent is feeling anxious, specific and easy-to-implement advice can be provided to help the parent feel at ease. Furthermore, the advice unit can learn from past advice results and parent feedback to improve the accuracy of advice. As a result, the parent can always take appropriate actions based on the latest information.

The health check system may further include a reminder unit. The reminder unit provides reminders for the parent to regularly perform health checks on the child. For example, notifications can be sent regularly to prompt health checks based on a schedule set by the parent. Additionally, the reminder unit can estimate the parent's emotion and adjust the content and timing of reminders based on the estimated emotion. For instance, if the parent is busy, the reminder unit can suggest postponing the reminder. Furthermore, the reminder unit can propose the optimal timing for reminders to the parent based on past reminder history. As a result, the parent can efficiently perform health checks on the child.

The health check system may further include a communication unit. The communication unit supports communication between the parent and the doctor. For example, a chat function can be provided for the parent to ask questions or consult with the doctor. Additionally, the communication unit can estimate the parent's emotion and adjust the content and method of communication based on the estimated emotion. For instance, if the parent is feeling anxious, the system can enable the parent to consult with the doctor quickly. Furthermore, the communication unit can provide appropriate advice or information to the parent based on past communication history. As a result, the parent can check the child's health condition with peace of mind and take appropriate actions.

The health check system may further include a data sharing unit. The data sharing unit provides a means for the parent to share the child's health data with other family members or medical institutions. For example, the parent can share the child's health data with the family so that all family members can grasp the child's health condition. Additionally, the data sharing unit can enable the parent to share data with medical institutions so that doctors can quickly grasp the child's health condition. Furthermore, the data sharing unit can estimate the parent's emotion and adjust the method and timing of data sharing based on the estimated emotion. For instance, if the parent is feeling anxious, data can be shared quickly and the parent can receive advice from the doctor.

The health check system may further include a prevention unit. The prevention unit provides information and advice for preventing the child's health problems. For example, methods for health management according to the season or schedules for vaccinations can be provided. Additionally, the prevention unit can estimate the parent's emotion and adjust the content and provision method of prevention information based on the estimated emotion. For instance, if the parent is feeling anxious, specific and easy-to-implement prevention methods can be provided to help the parent feel at ease. Furthermore, the prevention unit can learn from past prevention history and parent feedback to improve the accuracy of prevention information. As a result, the parent can always prevent the child's health problems based on the latest information.

The health check system may further include a feedback unit. The feedback unit provides a means for the parent to give feedback on the usability and improvements of the system. For example, the parent can send opinions regarding the usability and functions of the system. Additionally, the feedback unit can estimate the parent's emotion and adjust the content and method of feedback based on the estimated emotion. For instance, if the parent is dissatisfied, the system can respond quickly and reflect improvements. Furthermore, the feedback unit can analyze system improvements based on past feedback history and provide a user-friendly system for the parent. As a result, the parent can use the system with peace of mind.

The health check system may further include a customization unit. The customization unit provides a means for the parent to customize the system settings. For example, the parent can set the frequency and content of notifications and use the system in a way that suits them. Additionally, the customization unit can estimate the parent's emotion and propose customization options based on the estimated emotion. For instance, if the parent is feeling anxious, detailed notifications can be proposed to help the parent feel at ease. Furthermore, the customization unit can propose optimal settings for the parent based on past usage history. As a result, the parent can use the system in a way that suits them.

The health check system may further include an education unit. The education unit provides a means for the parent to learn knowledge about the child's health. For example, online courses and information regarding health management can be provided. Additionally, the education unit can estimate the parent's emotion and adjust the content and provision method of education based on the estimated emotion. For instance, if the parent is feeling anxious, specific and easy-to-implement information can be provided to help the parent feel at ease. Furthermore, the education unit can propose optimal learning content for the parent based on past learning history. As a result, the parent can always manage the child's health based on the latest information.

The following is a brief explanation of the processing flow of Example of the Embodiment.

Step 1: The reception unit receives images taken and audio recorded by the parent. The images and audio taken and recorded by the parent may include, for example, the child's complexion, facial expressions, and breathing sounds. The reception unit receives images and audio taken using a smartphone or tablet. In addition, the reception unit needs to satisfy conditions such as the resolution and format of the images and audio, and the sound quality. For example, it is desirable that the images are high-resolution and the audio is clear.

Step 2: The analysis unit analyzes the images and audio received by the reception unit. The analysis unit uses image analysis techniques to analyze the child's complexion, facial expressions, and body movements. For example, the system detects cases where the complexion is pale, the facial expression appears distressed, or the body is trembling. The analysis unit also uses speech recognition technology to detect breathing rhythms and abnormal sounds. For example, the system detects cases where breathing is rough, coughing is severe, or there are abnormal breathing sounds. As image analysis techniques, facial recognition technology and motion analysis technology can be used. As speech recognition technology, audio pattern recognition and abnormal sound detection technology can be used.

Step 3: The evaluation unit evaluates the health condition based on the result analyzed by the analysis unit. The evaluation unit evaluates the child's health condition based on the analysis result. For example, if the complexion is pale and breathing is rough, the system determines that the urgency is high. The evaluation unit needs to clarify the evaluation criteria and specific evaluation methods for health condition. For example, evaluation items and scoring methods can be set.

Step 4: The connection unit connects to a dedicated service desk based on the result evaluated by the evaluation unit. The connection unit can connect to a dedicated service desk as needed based on the evaluation result. For example, if the urgency is high, the system connects to an emergency service desk so that the parent can consult directly with a doctor. The connection unit needs to clarify the specific types and roles of dedicated service desks. For example, medical institutions and consultation desks can be set.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the aforementioned reception unit, analysis unit, evaluation unit, and connection unit is implemented by at least one of, for example, the smart device 14 and the data processing apparatus 12. For example, the reception unit is implemented by a control unit 46A of the smart device 14 and receives images taken and audio recorded by a parent. The analysis unit is implemented, for example, by a specific processing unit 290 of the data processing apparatus 12 and analyzes a child's complexion, facial expressions, breathing rhythms, and abnormal sounds using image analysis techniques and speech recognition technology. The evaluation unit is implemented, for example, by the specific processing unit 290 of the data processing apparatus 12 and evaluates a health condition based on the analysis result. The connection unit is implemented, for example, by the specific processing unit 290 of the data processing apparatus 12 and connects to a dedicated service desk based on the evaluation result. The correspondence between each unit and the apparatus or control unit is not limited to the examples described above and various modifications are possible.

### Second Embodiment

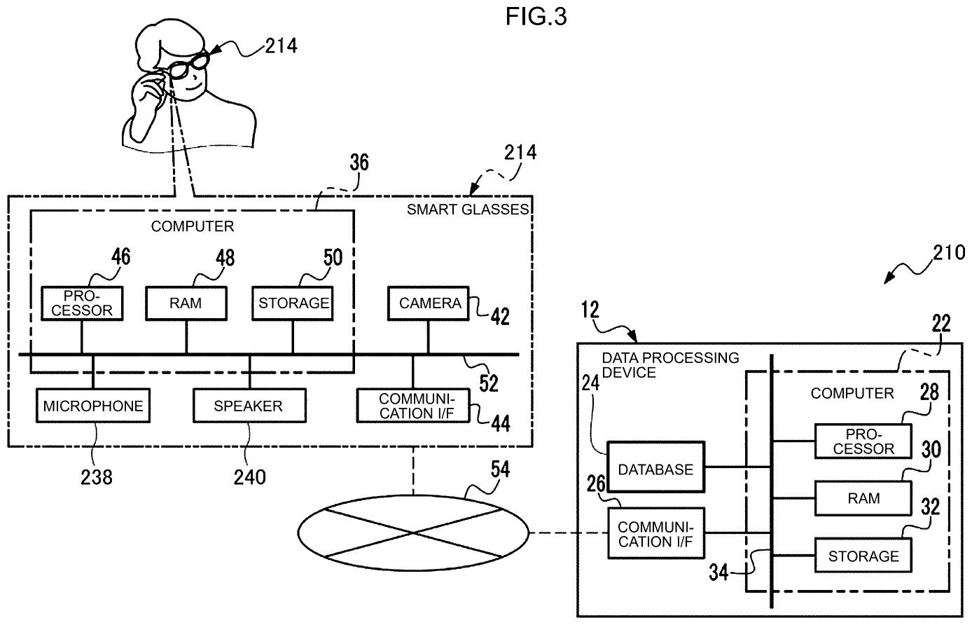

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

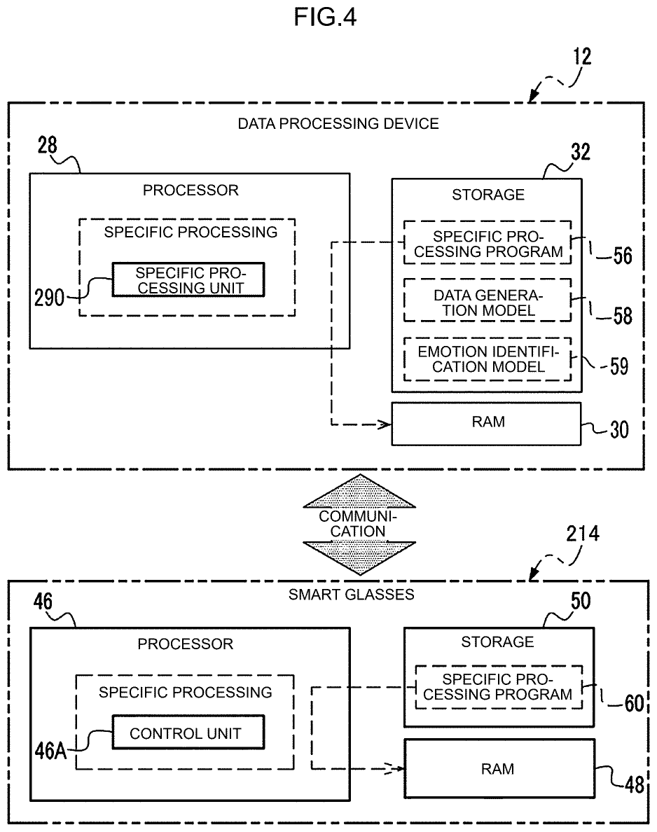

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the aforementioned reception unit, analysis unit, evaluation unit, and connection unit is implemented by at least one of, for example, the smart glasses 214 and the data processing apparatus 12. For example, the reception unit is implemented by a control unit 46A of the smart glasses 214 and receives images taken and audio recorded by a parent. The analysis unit is implemented, for example, by a specific processing unit 290 of the data processing apparatus 12 and analyzes a child's complexion, facial expressions, breathing rhythms, and abnormal sounds using image analysis techniques and speech recognition technology. The evaluation unit is implemented, for example, by the specific processing unit 290 of the data processing apparatus 12 and evaluates a health condition based on the analysis result. The connection unit is implemented, for example, by the specific processing unit 290 of the data processing apparatus 12 and connects to a dedicated service desk based on the evaluation result. The correspondence between each unit and the apparatus or control unit is not limited to the examples described above and various modifications are possible.

### Third Embodiment

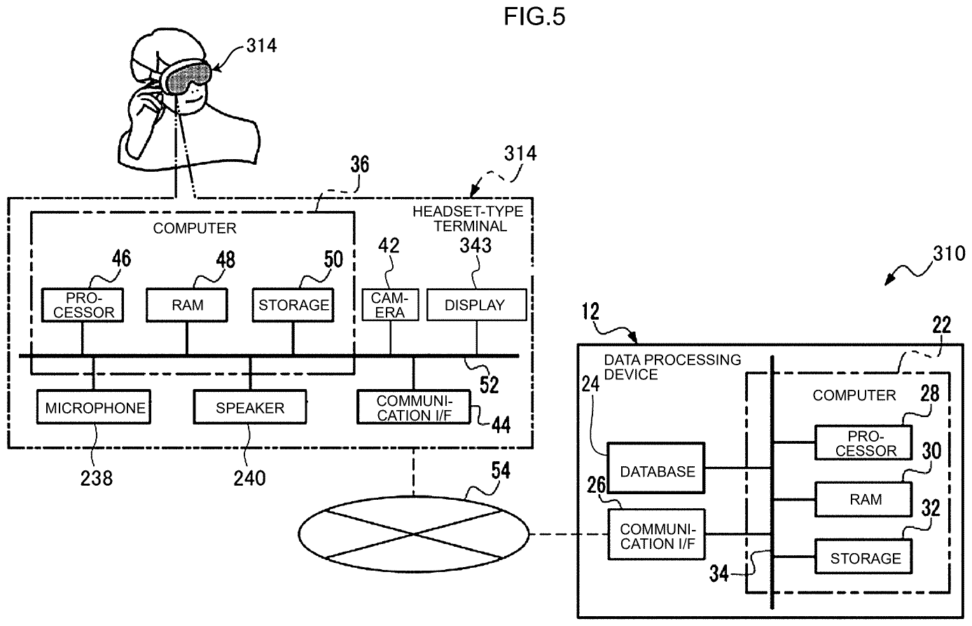

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

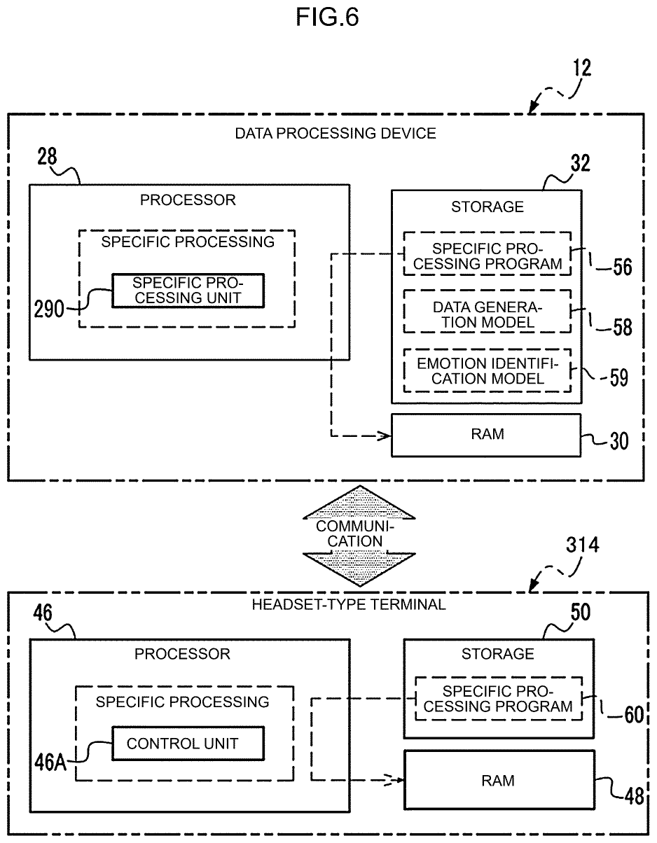

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the aforementioned reception unit, analysis unit, evaluation unit, and connection unit is implemented by at least one of, for example, the headset-type terminal 314 and the data processing apparatus 12. For example, the reception unit is implemented by a control unit 46A of the headset-type terminal 314 and receives images taken and audio recorded by a parent. The analysis unit is implemented, for example, by a specific processing unit 290 of the data processing apparatus 12 and analyzes a child's complexion, facial expressions, breathing rhythms, and abnormal sounds using image analysis techniques and speech recognition technology. The evaluation unit is implemented, for example, by the specific processing unit 290 of the data processing apparatus 12 and evaluates a health condition based on the analysis result. The connection unit is implemented, for example, by the specific processing unit 290 of the data processing apparatus 12 and connects to a dedicated service desk based on the evaluation result. The correspondence between each unit and the apparatus or control unit is not limited to the examples described above and various modifications are possible.

### Fourth Embodiment

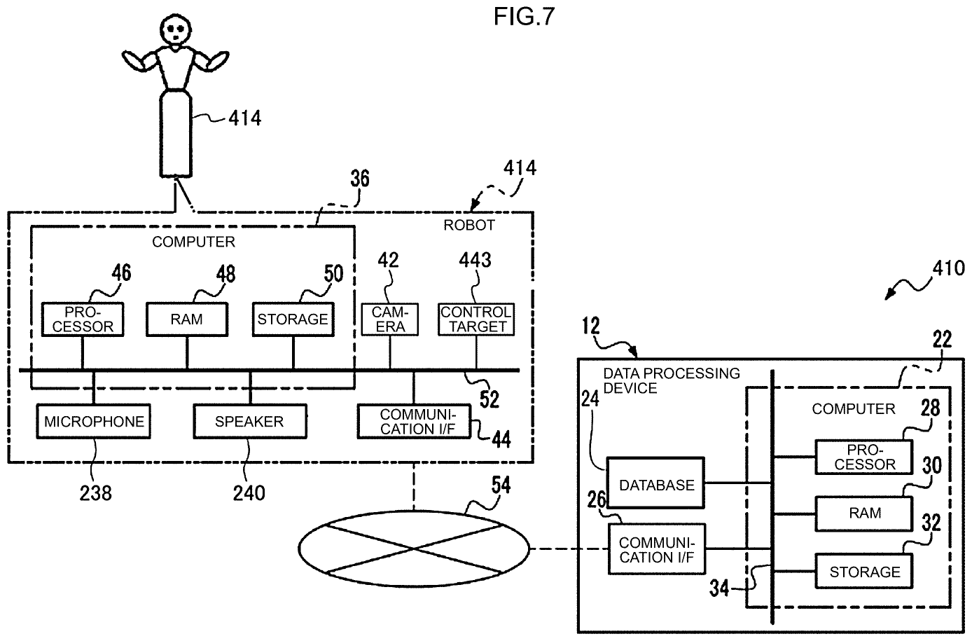

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

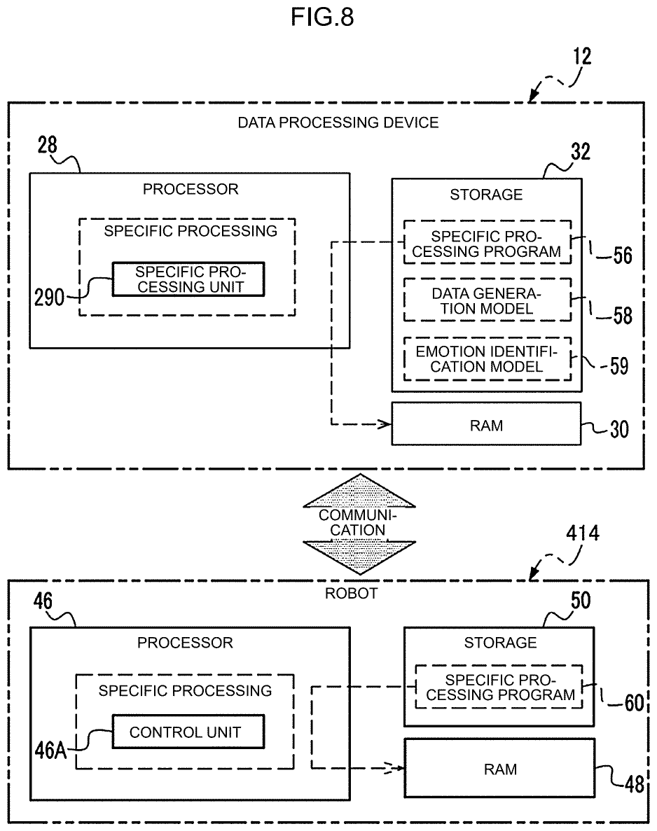

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the aforementioned reception unit, analysis unit, evaluation unit, and connection unit is implemented by at least one of, for example, the robot 414 and the data processing apparatus 12. For example, the reception unit is implemented by a control unit 46A of the robot 414 and receives images taken and audio recorded by a parent. The analysis unit is implemented, for example, by a specific processing unit 290 of the data processing apparatus 12 and analyzes a child's complexion, facial expressions, breathing rhythms, and abnormal sounds using image analysis techniques and speech recognition technology. The evaluation unit is implemented, for example, by the specific processing unit 290 of the data processing apparatus 12 and evaluates a health condition based on the analysis result. The connection unit is implemented, for example, by the specific processing unit 290 of the data processing apparatus 12 and connects to a dedicated service desk based on the evaluation result. The correspondence between each unit and the apparatus or control unit is not limited to the examples described above and various modifications are possible.

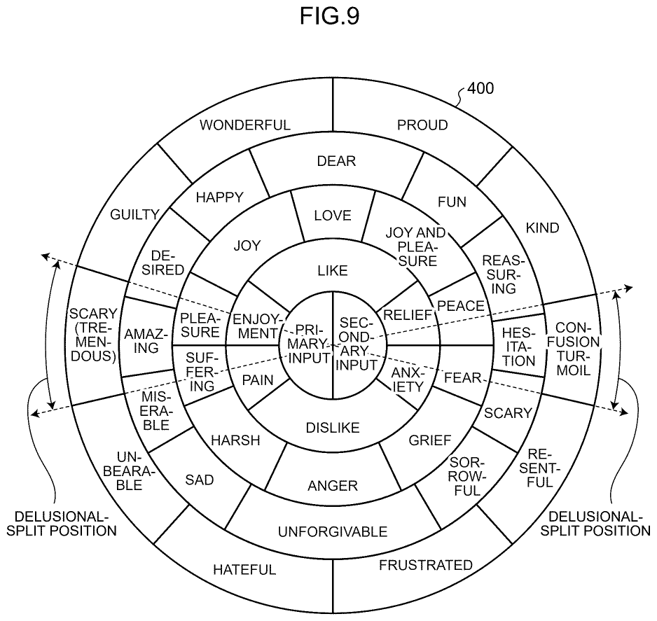

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

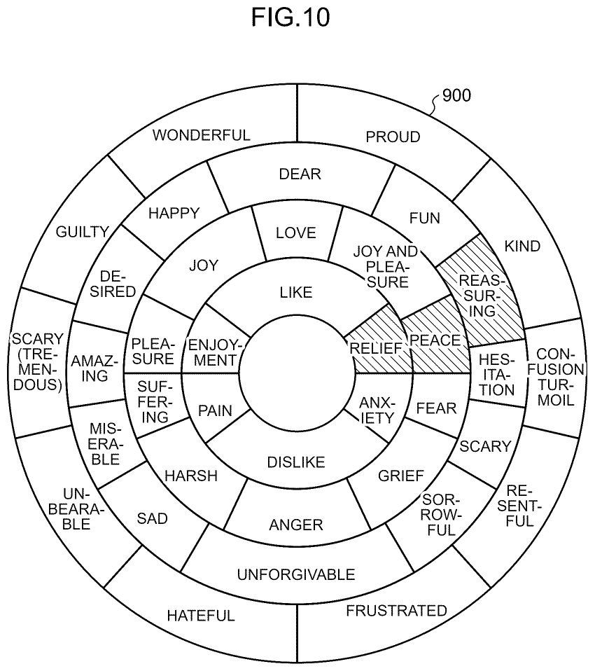

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.

### Supplementary Note 1

A system comprising: a reception unit configured to receive images and audio; an analysis unit configured to analyze the images and audio received by the reception unit; an evaluation unit configured to evaluate a health condition based on a result analyzed by the analysis unit; and a connection unit configured to connect to a specific service desk based on a result evaluated by the evaluation unit.

### Supplementary Note 2

The system according to Supplementary Note 1, further comprising a summarization unit configured to summarize a situation using generative AI.

### Supplementary Note 3

The system according to Supplementary Note 1, further comprising a learning unit configured to learn past analysis results or medical diagnosis results.

### Supplementary Note 4

The system according to Supplementary Note 1, wherein the reception unit is configured to receive images taken and audio recorded by a parent under specific conditions.

### Supplementary Note 5

The system according to Supplementary Note 1, wherein the analysis unit is configured to analyze a child's complexion, facial expressions, and body movements using specific image analysis techniques.

### Supplementary Note 6

The system according to Supplementary Note 1, wherein the analysis unit is configured to detect breathing rhythms and abnormal sounds using speech recognition technology.

### Supplementary Note 7

The system according to Supplementary Note 1, wherein the evaluation unit is configured to evaluate a health condition based on the analysis result.

### Supplementary Note 8

The system according to Supplementary Note 1, wherein the connection unit is configured to connect to a dedicated service desk based on the evaluation result.

### Supplementary Note 9

The system according to Supplementary Note 1, wherein the reception unit is configured to estimate a parent's emotion and adjust the timing of acquiring images and audio based on the estimated emotion.

### Supplementary Note 10

The system according to Supplementary Note 1, wherein the reception unit is configured to analyze a parent's past submission history of images and audio and select an appropriate acquisition method.

### Supplementary Note 11

The system according to Supplementary Note 1, wherein the reception unit is configured to perform filtering based on a parent's current living situation and areas of interest when acquiring images and audio.

### Supplementary Note 12

The system according to Supplementary Note 1, wherein the reception unit is configured to estimate a parent's emotion and determine the priority of images and audio to be acquired based on the estimated emotion.

### Supplementary Note 13

The system according to Supplementary Note 1, wherein the reception unit is configured to preferentially acquire highly relevant information based on a parent's geographic location when acquiring images and audio.

### Supplementary Note 14

The system according to Supplementary Note 1, wherein the reception unit is configured to analyze a parent's social media activity and acquire related information when acquiring images and audio.

### Supplementary Note 15

The system according to Supplementary Note 1, wherein the analysis unit is configured to estimate a parent's emotion and adjust the expression method of analysis based on the estimated emotion.

### Supplementary Note 16

The system according to Supplementary Note 1, wherein the analysis unit is configured to adjust the level of detail of analysis based on the importance of images and audio during analysis.

### Supplementary Note 17

The system according to Supplementary Note 1, wherein the analysis unit is configured to apply different analysis algorithms according to the category of images and audio during analysis.

### Supplementary Note 18

The system according to Supplementary Note 1, wherein the analysis unit is configured to estimate a parent's emotion and adjust the length of analysis based on the estimated emotion.

### Supplementary Note 19

The system according to Supplementary Note 1, wherein the analysis unit is configured to determine the priority of analysis based on the submission timing of images and audio during analysis.

### Supplementary Note 20

The system according to Supplementary Note 1, wherein the analysis unit is configured to adjust the order of analysis based on the relevance of images and audio during analysis.

### Supplementary Note 21

The system according to Supplementary Note 1, wherein the evaluation unit is configured to estimate a parent's emotion and adjust the evaluation criteria based on the estimated emotion.

### Supplementary Note 22

The system according to Supplementary Note 1, wherein the evaluation unit is configured to improve the accuracy of evaluation by considering the interrelationship between images and audio during evaluation.

### Supplementary Note 23

The system according to Supplementary Note 1, wherein the evaluation unit is configured to perform evaluation by considering attribute information of the submitter of images and audio during evaluation.

### Supplementary Note 24

The system according to Supplementary Note 1, wherein the evaluation unit is configured to estimate a parent's emotion and adjust the order of displaying evaluation results based on the estimated emotion.

### Supplementary Note 25

The system according to Supplementary Note 1, wherein the evaluation unit is configured to perform evaluation by considering the geographic distribution of images and audio during evaluation.

### Supplementary Note 26

The system according to Supplementary Note 1, wherein the evaluation unit is configured to improve the accuracy of evaluation by referring to related literature of images and audio during evaluation.

### Supplementary Note 27

The system according to Supplementary Note 1, wherein the connection unit is configured to estimate a parent's emotion and adjust the connection method based on the estimated emotion.

### Supplementary Note 28

The system according to Supplementary Note 1, wherein the connection unit is configured to adjust the level of detail of connection based on the importance of evaluation results during connection.

### Supplementary Note 29

The system according to Supplementary Note 1, wherein the connection unit is configured to apply different connection algorithms according to the category of evaluation results during connection.

### Supplementary Note 30

The system according to Supplementary Note 1, wherein the connection unit is configured to estimate a parent's emotion and determine the priority of connection based on the estimated emotion.

### Supplementary Note 31

The system according to Supplementary Note 1, wherein the connection unit is configured to adjust the order of connection based on the submission timing of evaluation results during connection.

### Supplementary Note 32

The system according to Supplementary Note 1, wherein the connection unit is configured to adjust the order of connection based on the relevance of evaluation results during connection.

### Supplementary Note 33

The system according to Supplementary Note 2, wherein the summarization unit is configured to estimate a parent's emotion and adjust the expression method of summarization based on the estimated emotion.

### Supplementary Note 34

The system according to Supplementary Note 2, wherein the summarization unit is configured to adjust the level of detail of summarization based on the importance of evaluation results during summarization generation.

### Supplementary Note 35

The system according to Supplementary Note 2, wherein the summarization unit is configured to apply different summarization algorithms according to the category of evaluation results during summarization generation.

### Supplementary Note 36

The system according to Supplementary Note 2, wherein the summarization unit is configured to estimate a parent's emotion and adjust the length of summarization based on the estimated emotion.

### Supplementary Note 37

The system according to Supplementary Note 2, wherein the summarization unit is configured to determine the priority of summarization based on the submission timing of evaluation results during summarization generation.

### Supplementary Note 38

The system according to Supplementary Note 2, wherein the summarization unit is configured to adjust the order of summarization based on the relevance of evaluation results during summarization generation.

### Supplementary Note 39

The system according to Supplementary Note 3, wherein the learning unit is configured to estimate a parent's emotion and select learning data based on the estimated emotion.

### Supplementary Note 40

The system according to Supplementary Note 3, wherein the learning unit is configured to optimize a learning algorithm by referring to past learning data during learning.

### Supplementary Note 41

The system according to Supplementary Note 3, wherein the learning unit is configured to estimate a parent's emotion and adjust the frequency of learning based on the estimated emotion.

### Supplementary Note 42

The system according to Supplementary Note 3, wherein the learning unit is configured to weight learning data based on the submission timing of images and audio during learning.

What is claimed is:

1. A system comprising:
circuitry configured to:
receive, from a client terminal via a packet-switched network, first sensor data comprising image frames and second sensor data comprising audio samples;
extract, from the image frames, a first feature vector by inputting the image frames into a convolutional neural network;
extract, from the audio samples, a second feature vector by inputting the audio samples into a recurrent neural network;
generate, based on the first feature vector and the second feature vector, a classification label by inputting the first feature vector and the second feature vector into an inference model, the classification label indicating an urgency level; and
transmit, to a remote terminal via the packet-switched network, connection data identifying a service endpoint corresponding to the classification label.

2. The system according to claim 1, wherein the circuitry is further configured to generate, by inputting the classification label and the first feature vector and the second feature vector into a Transformer-based language model, a natural language summary describing a condition associated with the classification label.

3. The system according to claim 1, wherein the circuitry is further configured to update weight parameters of the inference model by performing error backpropagation based on a loss function using historical classification labels and corresponding ground-truth labels as training data.

4. The system according to claim 1, wherein the first sensor data comprises RGB image data having a resolution of at least 1920 by 1080 pixels and 8-bit depth, and the second sensor data comprises pulse-code modulation audio data sampled at 16 kHz with 16-bit depth.

5. The system according to claim 1, wherein the convolutional neural network comprises a ResNet or EfficientNet architecture, and the first feature vector is a multidimensional tensor having at least 128 dimensions representing a plurality of visual attributes extracted from the image frames.

6. The system according to claim 1, wherein the recurrent neural network comprises a long short-term memory network, and the second feature vector represents at least one of a temporal rhythm pattern or an abnormality score computed from Mel-frequency cepstral coefficient features extracted from the audio samples.

7. The system according to claim 1, wherein the circuitry is further configured to:
compute a weighted score by applying weighting parameters to the first feature vector and the second feature vector; and
generate the classification label by comparing the weighted score against a plurality of threshold values, each threshold value corresponding to a respective urgency level.

8. The system according to claim 1, wherein the connection data comprises at least one of a session identifier, an authentication credential, or a communication channel address, and the service endpoint is selected from a plurality of service endpoints based on the urgency level indicated by the classification label.

9. The system according to claim 1, wherein the circuitry is further configured to:
estimate an emotion of a user associated with the client terminal by applying a facial expression classifier to a facial image received from the client terminal; and
adjust a timing of the receive based on the estimated emotion, such that when the estimated emotion indicates a high-urgency state, the circuitry prompts immediate acquisition of the first sensor data and the second sensor data.

10. The system according to claim 1, wherein the circuitry is further configured to analyze a submission history associated with the client terminal, the submission history comprising timestamps and data format information of previously received sensor data, and select an acquisition method for the first sensor data and the second sensor data based on the submission history.

11. The system according to claim 1, wherein the circuitry is further configured to estimate an emotion of a user associated with the client terminal and adjust a display format of the classification label based on the estimated emotion, such that when the estimated emotion indicates a high-stress state, the circuitry generates a simplified output, and when the estimated emotion indicates a low-stress state, the circuitry generates a detailed output including the first feature vector and the second feature vector.

12. The system according to claim 1, wherein the circuitry is further configured to compute an importance score for the first sensor data and the second sensor data based on at least one of an information value or a content urgency, and adjust a granularity of the extract based on the importance score.

13. The system according to claim 1, wherein the circuitry is further configured to apply different analysis algorithms to the image frames and the audio samples according to a category of the first sensor data and the second sensor data, the category being determined by at least one of a content type or a data format.

14. The system according to claim 1, wherein the circuitry is further configured to determine a priority of the first sensor data and the second sensor data based on a submission timestamp associated with each of the first sensor data and the second sensor data, such that more recently submitted sensor data is processed before earlier submitted sensor data.

15. The system according to claim 1, wherein the circuitry is further configured to compute a relevance score between the first sensor data and the second sensor data based on a cosine similarity between the first feature vector and the second feature vector, and adjust an order of processing based on the relevance score.

16. The system according to claim 1, wherein the circuitry is further configured to receive geographic location information from the client terminal, and adjust the receive by selecting an acquisition mode for the first sensor data and the second sensor data based on the geographic location information.

17. The system according to claim 1, wherein the circuitry is further configured to record connection history comprising the classification label, the service endpoint, and a response result, and optimize a subsequent selection of the service endpoint based on the connection history.

18. A system comprising:
circuitry configured to:
receive, from a client terminal via a packet-switched network, first sensor data comprising RGB image frames and second sensor data comprising pulse-code modulation audio samples;
perform preprocessing on the RGB image frames comprising noise removal, region extraction, and color space conversion, and perform preprocessing on the pulse-code modulation audio samples comprising silent segment removal, spectrogram conversion, and Mel-frequency cepstral coefficient extraction;
extract, from the preprocessed RGB image frames, a first multidimensional tensor by inputting the preprocessed RGB image frames into a convolutional neural network, the first multidimensional tensor representing a plurality of visual attributes;
extract, from the preprocessed pulse-code modulation audio samples, a second multidimensional tensor by inputting the preprocessed pulse-code modulation audio samples into a long short-term memory network, the second multidimensional tensor representing at least one of a temporal rhythm pattern or an abnormality score;
compute a weighted score by applying weighting parameters to the first multidimensional tensor and the second multidimensional tensor;
generate, based on the weighted score, a classification label by comparing the weighted score against a plurality of threshold values, each threshold value corresponding to a respective urgency level;
generate, by inputting the classification label, the first multidimensional tensor, and the second multidimensional tensor into a Transformer-based language model, a natural language summary; and
transmit, to a remote terminal via the packet-switched network, connection data identifying a service endpoint corresponding to the classification label and the natural language summary.

19. The system according to claim 18, wherein the circuitry is further configured to update weight parameters of the convolutional neural network and the long short-term memory network by performing error backpropagation using a cross-entropy loss function, the update being based on training data comprising historical sensor data and corresponding ground-truth labels, the training data augmented by at least one of image rotation, brightness adjustment, or audio pitch shifting.

20. A method performed by a system comprising circuitry, the method comprising:
receiving, from a client terminal via a packet-switched network, first sensor data comprising image frames and second sensor data comprising audio samples;
extracting, from the image frames, a first feature vector by inputting the image frames into a convolutional neural network;
extracting, from the audio samples, a second feature vector by inputting the audio samples into a recurrent neural network;
generating, based on the first feature vector and the second feature vector, a classification label by inputting the first feature vector and the second feature vector into an inference model, the classification label indicating an urgency level; and
transmitting, to a remote terminal via the packet-switched network, connection data identifying a service endpoint corresponding to the classification label.
